SEQUENCE LISTING (8 sequences)

SEQ ID NO: 1 - DNA, 1707 bp, Paramecium bursaria Chlorella Virus 1
  CDS (1)..(1707)
  ref <308>: DATABASE ACCESSION NUMBER: PB42580
  ref <309>: DATABASE ENTRY DATE: 1995-12-24
  ref <313>: RELEVANT RESIDUES IN SEQ ID NO: (50903)..(52609)
atgggtaaaaatataatcataatggtttcgtggtacaccatcataacttcaaatctaatcgcggttggaggagcctctctaatcttggctccggcaattactgggtatgttctacattggaatattgctctctcgacaatctggggagtatcagcttatggtattttcgtttttgggtttttccttgcacaagttttattttcagaactgaacaggaaacgtcttcgcaagtggatttctctcagacctaagggttggaatgatgttcgtttggctgtgatcattgctggatatcgcgaggatccttatatgttccagaagtgcctcgagtctgtacgtgactctgattatggcaacgttgcccgtctgatttgtgtgattgacggtgatgaggacgatgatatgaggatggctgccgtttacaaggcgatctacaatgataatatcaagaagcccgagtttgttctgtgtgagtcagacgacaaggaaggtgaacgcatcgactctgatttctctcgcgacatttgtgtcctccagcctcatcgtggaaaacgggagtgtctttatactgggtttcaacttgcaaagatggaccccagtgtcaatgctgtcgttctgattgacagcgataccgttctcgagaaggatgctattctggaagttgtatacccacttgcatgcgatcccgagatccaagccgttgcaggtgagtgtaagatttggaacacagacactcttttgagtcttctcgtcgcttggcggtactattctgcgttttgtgtggagaggagtgcccagtcttttttcaggactgttcagtgcgttggggggccactgggtgcctacaagattgatatcattaaggagattaaggacccctggatttcccagcgctttcttggtcagaagtgtacttacggtgacgaccgccggctaaccaacgagatcttgatgcgtggtaaaaaggttgtgttcactccatttgctgttggttggtctgacagtccgaccaatgtgtttcggtacatcgttcagcagacccgctggagtaagtcgtggtgccgcgaaatttggtacaccctcttcgccgcgtggaagcacggtttgtctggaatttggctggcctttgaatgtttgtatcaaattacatacttcttcctcgtgatttacctcttttctcgcctagccgttgaggccgaccctcgcgcccagacagccacggtgattgtgagcaccacggttgcattgattaagtgtgggtatttttcattccgagccaaggatattcgggcgttttactttgtgctttatacatttgtttactttttctgtatgattccggccaggattactgcaatgatgacgctttgggacattggctggggtactcgcggtggaaacgagaagccttccgttggcacccgggtcgctctgtgggcaaagcaatatctcattgcatatatgtggtgggccgcggttgttggcgctggagtttacagcatcgtccataactggatgttcgattggaattctctttcttatcgttttgctttggttggtatttgttcttacattgtttttattgttattgtgctggtggtttatttcaccggcaaaattacgacttggaatttcacgaagcttcagaaggagctaatcgaggatcgcgttctgtacgatgcaactaccaatgctcagtctgtgtga

SEQ ID NO: 2 - protein, 568 aa, Paramecium bursaria Chlorella Virus 1
MGKNIIIMVSWYTIITSNLIAVGGASLILAPAITGYVLHWNIALSTIWGVSAYGIFVFGFFLAQVLFSELNRKRLRKWISLRPKGWNDVRLAVIIAGYREDPYMFQKCLESVRDSDYGNVARLICVIDGDEDDDMRMAAVYKAIYNDNIKKPEFVLCESDDKEGERIDSDFSRDICVLQPHRGKRECLYTGFQLAKMDPSVNAVVLIDSDTVLEKDAILEVVYPLACDPEIQAVAGECKIWNTDTLLSLLVAWRYYSAFCVERSAQSFFRTVQCVGGPLGAYKIDIIKEIKDPWISQRFLGQKCTYGDDRRLTNEILMRGKKVVFTPFAVGWSDSPTNVFRYIVQQTRWSKSWCREIWYTLFAAWKHGLSGIWLAFECLYQITYFFLVIYLFSRLAVEADPRAQTATVIVSTTVALIKCGYFSFRAKDIRAFYFVLYTFVYFFCMIPARITAMMTLWDIGWGTRGGNEKPSVGTRVALWAKQYLIAYMWWAAVVGAGVYSIVHNWMFDWNSLSYRFALVGICSYIVFIVIVLVVYFTGKITTWNFTKLQKELIEDRVLYDATTNAQSV

SEQ ID NO: 3 - DNA, 1707 bp, Artificial Sequence
  Synthetic sequence encoding Paramecium bursaria Chlorella Virus Hyaluronansynthase protein
atgggtaagaacattatcattatggtgtcctggtacacaattattacaagtaatctcatcgcagttggtggtgcatctcttattctcgctccagctatcactggatatgttcttcactggaacatcgccctctcaactatttggggagtttccgcatatggtatttttgttttcgggttctttttggctcaggttctgttctcagagctcaatcgtaagagactcaggaagtggattagccttagaccaaaggggtggaatgacgttcgtctcgctgtcattatcgctggctaccgtgaagatccttacatgtttcaaaagtgcttggaatcagttagggatagtgattatggcaacgtcgctagactgatctgtgtgattgatggagatgaggacgacgatatgaggatggcagctgtttataaggctatctataatgataacattaagaagcctgaatttgttctttgcgagtctgatgacaaggaaggagaacggattgattcagatttctcacgtgatatctgcgttctccaacctcatcgtgggaagcgtgaatgtctttatacaggtttccaactcgccaaaatggacccatcagtgaacgctgtggttcttatcgatagtgatactgtgctggagaaagatgctatcttggaggttgtttaccctcttgcctgtgatcctgaaattcaagctgtggctggagagtgcaagatctggaacacagatactcttctttctctgcttgtcgcatggagatattactccgcattctgtgtggagaggagcgctcaatcctttttccgtaccgttcaatgcgttggtggtcctttgggagcttacaaaattgatatcatcaaggagattaaggacccatggattagtcaaaggtttcttggtcagaagtgcacttatggcgatgatcgtagattgactaacgaaatccttatgaggggcaagaaagtcgtttttactccatttgctgtcggatggtctgattcacctacaaatgttttccgttatattgtgcaacaaacacgttggagtaagagctggtgtagggagatctggtacactttgttcgctgcttggaagcacgggcttagcggaatttggcttgcttttgaatgcctttaccagattacatactttttcttggtgatctatttgttttcacgtcttgccgtcgaggctgaccctagagcacagactgcaactgtgattgtttctactacagtcgcacttattaagtgtggctatttcagttttagagcaaaagatattagagccttctattttgttttgtacacatttgtttatttcttttgcatgattccagctcgtattaccgctatgatgaccttgtgggacatcggatggggaactagaggtggtaacgaaaagccttctgtgggaacaagggtggccctttgggcaaaacaatatctcatcgcctacatgtggtgggccgctgtcgttggtgccggagtgtactcaatcgttcataactggatgtttgactggaactctttgagctatcgtttcgctcttgtgggtatttgttcttacattgttttcatcgtgattgtgctcgttgtgtatttcactggtaaaatcacaacctggaatttcactaaacttcaaaaggaattgattgaagacagggttctgtatgatgctactaccaacgcccagtcagtttaa

SEQ ID NO: 4 - DNA, 1260 bp, Paramecium bursaria Chlorella Virus 1
  CDS (62)..(1228)
  ref <308>: DATABASE ACCESSION NUMBER: U42580.4
  ref <309>: DATABASE ENTRY DATE: 2004-09-20
  ref <313>: RELEVANT RESIDUES IN SEQ ID NO: (291749.)..(292918)
atcaacgtgatttatattttaaacaaagaccattcacatctttagtacttaattaattataatgtcacgaatcgcagtcgttggttgtggttacgtcggaaccgcttgtgcagtacttcttgctcaaaaaaacgaagtcatcgtgcttgatattagcgaagaccgtgttcaactaatcaagaacaagaagagtccaatcgaggacaaggaaatcgaagagtttctcgaaacgaaagacctgaacctgaccgcgacgactgacaaggttcttgcatacgaaaacgccgaatttgtcatcatcgcaaccccgactgactatgacgtggttactaggtattttaacacgaaatctgtggaaaacgtcattggggacgtgatcaaaaatacacagacccatccaactatcgtgattaaatctaccatccccattggatttgttgataaggttcgtgagcaattcgactaccaaaatatcattttctccccagaatttctgcgtgaaggtagagccttgtatgataatctctacccatcccgtatcatcgtaggagatgattcccccattgcgcttaagttcgcaaaccttctcgttgaaggttctaaaactccgcttgcccctgtcctgacgatgggaactcgcgaagccgaggccgtcaaactattctctaacacgtatcttgcaatgcgagttgcatacttcaacgaactagatacattcgcaatgtctcacggtatgaatgcgaaagaaatcattgatggtgtgactctggagcctcgcattggtcaggggtactcaaacccttcgttcggttatggagcttattgctttccaaaggatacgaagcaactgctggctaatttcgagggagtgcctcaagatatcatcggagcaattgtagaatcaaatgagactcgcaaggaagtgattgtgagtgaagtagaaaatcgtttccccacgactgttggtgtgtataagctcgccgctaaagcgggttctgataattttcggagttctgcaattgtagacataatggagcgacttgcaaacaagggttatcacattaagattttcgaaccaactgtggaacaattcgaaaactttgaagttgataacaacctgacaacatttgcgactgagagcgatgtaattatcgcaaacagagttcccgttgaacatcgcattctctttggtaaaaaattaatcacacgtgatgtatatggcgataactaaaatgttttcaatatgatgttgttaatgat

SEQ ID NO: 5 - protein, 389 aa, Paramecium bursaria Chlorella Virus 1
MSRIAVVGCGYVGTACAVLLAQKNEVIVLDISEDRVQLIKNKKSPIEDKEIEEFLETKDLNLTATTDKVLAYENAEFVIIATPTDYDVVTRYFNTKSVENVIGDVIKNTQTHPTIVIKSTIPIGFVDKVREQFDYQNIIFSPEFLREGRALYDNLYPSRIIVGDDSPIALKFANLLVEGSKTPLAPVLTMGTREAEAVKLFSNTYLAMRVAYFNELDTFAMSHGMNAKEIIDGVTLEPRIGQGYSNPSFGYGAYCFPKDTKQLLANFEGVPQDIIGAIVESNETRKEVIVSEVENRFPTTVGVYKLAAKAGSDNFRSSAIVDIMERLANKGYHIKIFEPTVEQFENFEVDNNLTTFATESDVIIANRVPVEHRILFGKKLITRDVYGDN

SEQ ID NO: 6 - DNA, 1170 bp, Artificial Sequence
  Synthetic sequence encoding a Paramecium bursaria Chlorella Virus protein having the activity of a UDP-Glc-DH
atgtctcgcatagctgttgtaggatgtggctatgtgggaactgcatgtgcggttctacttgctcaaaagaacgaagttattgtgcttgatattagtgaagaccgtgttcaacttattaagaacaagaagtctcctattgaggataaggaaatcgaagagttcttggaaacaaaggatcttaatcttactgcgactacagataaggttcttgcctacgagaacgctgagtttgtgataatcgctacaccaaccgattacgacgttgtgactcgatatttcaataccaaatccgtggaaaacgttataggagatgttatcaagaacactcaaacccaccctactatcgtcatcaagtccacaattcccatcggtttcgttgataaggtcagagagcagtttgattatcaaaacattatcttctcacctgagttcttaagggagggtcgtgctctctacgataatttgtatccgtcccgtattatcgttggcgacgattctcctatcgctctcaagttcgcaaatctcttagttgagggtagtaagacccctttggctcctgttttgacaatgggaaccagagaagcagaagctgtcaagctattctctaatacctaccttgccatgagggtagcatactttaacgaacttgatacatttgctatgtcgcatggtatgaatgccaaggagattatagatggtgtcactttagagcccaggatcggtcaaggatattctaacccatcattcggctatggagcttactgctttcctaaggacactaagcagttgctggcaaacttcgagggagttcctcaagacatcataggcgctattgtggagtcaaacgaaacaaggaaagaggtgatagttagtgaggtagagaatcgtttcccaacgacagtcggtgtttacaaactggcagctaaagctggtagcgataacttcaggtcaagtgctattgtcgacatcatggaacgcctggctaacaaaggttaccacattaagatctttgagccaactgtagagcagttcgaaaatttcgaagttgacaataacttgacaacgtttgctactgagtcagacgttattatcgcaaatcgtgtccctgtggaacatagaatcctatttggaaagaagctcattaccagagatgtttacggtgataattaa

SEQ ID NO: 7 - DNA, 48 bp, Artificial Sequence
  Synthetic oligonucleotide
tcgacaggcctggatccttaattaaactagtctcgaggagctcggtac

SEQ ID NO: 8 - DNA, 40 bp, Artificial Sequence
  Synthetic oligonucleotide
cgagctcctcgagactagtttaattaaggatccaggcctg

(19) United States
(12) Patent Application Publication
(10) Pub. No.: US 2008/0250533 A1
(43) Pub. Date: Oct. 9, 2008

(54) Plants With an Increased Production of Hyaluronan II

(76) Inventor: Claus Frohberg, Kleinmachnow (DE)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 12/089,299

(22) PCT Filed: Oct. 5, 2006
(86) PCT No.: PCT/EP06/09774

§ 371 Date: Apr. 4, 2008

Related U.S. Application Data
(60) Provisional application No. 60/725,183, filed on Oct. 11, 2005.

(30) Foreign Application Priority Data
Oct. 5, 2005 (EP) 05090278.2

(51) Int. Cl. C12N 15/87; C12N 5/10; C12P 19/26; A01H 5/00

(52) U.S. Cl. 800/284; 435/419; 800/298; 435/84; 800/305; 800/307; 800/309; 800/310; 800/312; 800/313; 800/315; 800/318; 800/317.1; 800/317.2; 800/317.4; 800/320.2; 800/320.3; 800/320

(57) ABSTRACT

The present invention relates to plant cells and plants which synthesize an increased amount of hyaluronan, and to methods for preparing such plants, and also to methods for preparing hyaluronan with the aid of these plant cells or plants. Here, plant cells or genetically modified plants according to the invention have hyaluronan synthase activity and additionally an increased UDP-glucose dehydrogenase (UDP-Glc-DH) activity compared to wild-type plant cells or wild-type plants. The present invention furthermore relates to the use of plants having increased hyaluronan synthesis for preparing hyaluronan and food or feedstuff containing hyaluronan.

The present invention relates to plant cells and plants which synthesize an increased amount of hyaluronan, and to methods for preparing such plants, and also to methods for preparing hyaluronan with the aid of these plant cells or plants. Here, plant cells or genetically modified plants according to the invention have hyaluronan synthase activity and additionally an increased UDP-glucose dehydrogenase (UDP-Glc-DH) activity compared to wild-type plant cells or wild-type plants. The present invention furthermore relates to the use of plants having increased hyaluronan synthesis for preparing hyaluronan and food or feedstuff containing hyaluronan.

Hyaluronan is a naturally occurring unbranched, linear mucopolysaccharide (glucosaminoglucan) which is constructed of alternating molecules of glucuronic acid and N-acetyl-glucosamine. The basic building block of hyaluronan consists of the disaccharide glucuronic acid-beta-1,3-N-acetyl-glucosamine. In hyaluronan, these repeating units are attached to one another via beta-1,4 linkages.

In pharmacy, use is frequently made of the term hyaluronic acid. Since hyaluronan is in most cases present as a polyanion and not as the free acid, hereinbelow, the term hyaluronan is preferably used, but each term is to be understood as embracing both molecular forms.

Hyaluronan has unusual physical chemical properties, such as, for example, properties of polyelectrolytes, viscoelastic properties, a high capacity to bind water, properties of gel formation, which, in addition to further properties of hyaluronan, are described in a review article by Lapcik et al. (1998, Chemical Reviews 98(8), 2663-2684).

Hyaluronan is a component of extracellular connective tissue and bodily fluids of vertebrates. In humans, hyaluronic acid is synthesized by the cell membrane of all body cells, especially mesenchymal cells, and ubiquitously present in the body with a particularly high concentration in the connective tissues, the extracellular matrix, the particularly high concentration in the connective tissues, the extracellular matrix, the umbilical cord, the joint fluid, the cartilaginous tissue, the skin and the vitreous body of the eye (Bernhard Gebauer, 1998, Inaugural-Dissertation, Virchow-Klinikum Medizinische Fakultät Charité der Humboldt Universität zu Berlin; Fraser et al., 1997, Journal of Internal Medicine 242, 27-33).

Recently, hyaluronan was also found in animal non-vertebrate organisms (molluscs) (Volpi and Maccari, 2003, Biochimie 85, 619-625).

Furthermore, some pathogenic gram-positive bacteria (Streptococcus group A and C) and gram-negative bacteria (Pasteurella) synthesize hyaluronan as exopolysaccharides which protect these bacteria against attack by the immune system of their host, since hyaluronan is a non-immunogenic substance.

Viruses which infect single-cell green algae of the genus Chlorella, some of which are present as endosymbionts in Paramecium species, bestow upon the single-cell green algae the ability to synthesize hyaluronan after infection by the virus (Graves et al., 1999, Virology 257, 15-23). However, the ability to synthesize hyaluronan is not a feature which characterizes the algae in question. The ability of the algae to synthesize hyaluronan is mediated by an infection with a virus whose genome has a sequence coding for hyaluronan synthase (DeAngelis, 1997, Science 278, 1800-1803). Furthermore, the virus genome contains sequences coding for a UDP-glucose dehydrogenase (UDP-Glc-DH). UDP-Glc-DH catalyses the synthesis of UDP-glucaronic acid used by the hyaluronan synthase as a substrate (DeAngelis et al., 1997, Science 278, 1800-1803, Graves et al., 1999, Virology 257, 15-23). The role of the expression of UDP-Glc-DH in virus-infected Chlorella cells for the hyaluronan synthesis, and whether they are required for hyaluronan synthesis, is not known.

Naturally occurring plants themselves do not have any nucleic acids in their genome which code for proteins catalyzing the synthesis of hyaluronan and, although a large number of plant carbohydrates have been described and characterized, it has hitherto not been possible to detect hyaluronan or molecules related to hyaluronan in non-infected plants (Graves et al., 1999, Virology 257, 15-23).

The catalysis of the hyaluronan synthesis is effected by a single membrane-integrated or membrane-associated enzyme, hyaluronan synthase. The hyaluronan synthases which have hitherto been studied can be classified into two groups: hyaluronan synthases of Class I and hyaluronan synthases of Class II (DeAngelis, 1999, CMLS, Cellular and Molecular Life Sciences 56, 670-682).

The hyaluronan synthases of vertebrates are further distinguished by the identified isoenzymes. The different isoenzymes are referred to in the order of their identification using Arabic numbers (for example, hsHAS1, hsHAS2, hsHAS3).

The mechanism of the transfer of synthesized hyaluronan molecules across the cytoplasma membrane into the medium surrounding the cell has not yet been fully elucidated. Earlier hypotheses assumed that transport across the cell membrane was effected by hyaluronan synthase itself. However, more recent results indicate that the transport of hyaluronan molecules across the cytoplasma membrane takes place by energy-dependent transport via transport proteins responsible for this action. Thus, Streptococcus strains were generated by mutation in which the synthesis of an active transport protein was inhibited. These strains synthesized less hyaluronan than corresponding wild-type bacteria strains (Ouskova et al., 2004, Glycobiology 14(10), 931-938). In human fibroblasts, it was possible to demonstrate, with the aid of agents specifically inhibiting known transport proteins, that it is possible to reduce both the amount of hyaluronan produced and the activity of hyaluronan synthases (Prehm and Schumacher, 2004, Biochemical Pharmacology 68, 1401-1410). In which amount, if at all, transport proteins capable of transporting hyaluronan are present in plants is not known.

The unusual properties of hyaluronan offer a wealth of possibilities for application in various fields, such as, for example, pharmacy, the cosmetics industry, in the production of food and feed, in technical applications (for example as lubricants), etc. The most important applications where hyaluronan is currently being used are in the medicinal and cosmetics field (see, for example, Lapcik et al., 1998, Chemical Reviews 98(8), 2663-2684, Goa and Benfield, 1994, Drugs 47(3), 536-566).

In the medical field, hyaluronan-containing products are currently used for the intraarticular treatment of arthrosis and in ophthalmics used for eye surgery. Hyaluronan is also used for treating joint disorders in racehorses. In addition, hyaluronic acid is a component of some rhinologics which, for example in the form of eye drops and nasalia, serve to moisten dry mucous membranes. Hyaluronan-containing solutions for injection are used as analgesics and antirheumatics. Patches comprising hyaluronan or derivatized hyaluronan are employed in wound healing. As dermatics, hyaluronan-containing gel implants are used for correcting skin deformations in plastic surgery. For pharmacological applications, preference is given to using hyaluronan having a high molecular weight.

In cosmetic medicine, hyaluronan preparations are among the most suitable skin filler materials. By injecting hyaluronan, for a limited period of time, it is possible to smooth wrinkles or to increase the volume of lips.

In cosmetic products, in particular in skin creams and lotions, hyaluronan is frequently used as moisturizer by virtue of its high water-binding capacity.

Furthermore, hyaluronan-containing preparations are sold as so-called nutraceuticals (food supplements) which can also be used in animals (for example dogs, horses) for the prophylaxis and alleviation of arthrosis.

Hyaluronan used for commercial purposes is currently isolated from animal tissues (cockscombs) or prepared fermentatively using bacterial cultures.

U.S. Pat. No. 4,141,973 describes a process for isolating hyaluronan from cockscombs or alternatively from umbilical cords. In addition to hyaluronan, animal tissues (for example cockscombs, umbilical cords) also contain further mucopolysaccharides related to hyaluronan, such as chondroitin sulfate, dermatan sulfate, keratan sulfate, heparan sulfate and heparin. Furthermore, animal organisms contain proteins (hyaladherins) which bind specifically to hyaluronan and which are required for the most different functions in the organism, such as, for example, the degradation of hyaluronan in the liver, the function of hyaluronan as lead structure for cell migration, the regulation of endocytosis, the anchoring of hyaluronan on the cell surface or the formation of hyaluronan networks (Turley, 1991, Adv Drug Delivery Rev 7, 257 ff., Laurent and Fraser, 1992, FASEB J. 6, 183 ff.; Stamenkovic and Aruffo, 1993, Methods Enzymol. 245, 195 ff; Knudson and Knudson, 1993, FASEB 7, 1233 ff.).

The Streptococcus strains used for the bacterial production of hyaluronan are exclusively pathogenic bacteria. During cultivation, too, these bacteria produce (pyrogenic) exotoxins and hemolysins (streptolysin, (in particular alpha- and beta-hemolysin) (Kilian, M.: Streptococcus and Enterococcus. In: Medical Microbiology. Greenwood, D.; Slack, R C A; Peutherer, J. F. (Eds.). Chapter 16. Churchill Livingstone, Edinburgh, UK: pp. 174-188, 2002, ISBN 0443070776) which are released into the culture medium. This renders purification and isolation of the hyaluronan prepared with the aid of Streptococcus strains more difficult. In particular for pharmaceutical application, the presence of exotoxins and hemolysins in the preparation is a problem.

U.S. Pat. No. 4,801,539 describes the preparation of hyaluronan by fermentation of a mutagenized bacterial strain (Streptococcus zooedemicus). The mutagenized bacteria strain used no longer synthesizes beta-hemolysin. The yield achieved was 3.6 g of hyaluronan per liter of culture.

EP 0694616 describes a method for cultivating Streptococcus zooedemicus or Streptococcus equi, where, under the culture conditions employed, no streptolysin, but increased amounts of hyaluronan are synthesized. The yield achieved was 3.5 g of hyaluronan per liter of culture.

During cultivation, Streptococcus strains release the enzyme hyaluronidase into the culture medium, as a consequence of which, in this production system, too, the molecular weight is reduced during purification. The use of hyaluronidase-negative Streptococcus strains or of methods for the production of hyaluronan where the production of hyaluronidase during cultivation is inhibited are described in U.S. Pat. No. 4,782,046. The yield achieved was up to 2.5 g of hyaluronan per liter of culture, and the maximum mean molecular weight achieved was 3.8×10⁶ Da, at a molecular weight distribution of from 2.4×10⁶ to 4.0×10⁶.

US 20030175902 and WO 03 054163 describe the preparation of hyaluronan with the aid of heterologous expression of a hyaluronan synthase from Streptococcus equisimilis in Bacillus subtilis. To achieve the production of sufficient amounts of hyaluronan, in addition to heterologous expression of a hyaluronan synthase, simultaneous expression of a UDP-glucose dehydrogenase in the Bacillus cells is also required. US 20030175902 and WO 03 054163 do not state the absolute amount of hyaluronan obtained in the production with the aid of Bacillus subtilis. The maximum mean molecular weight achieved was about 4.2×10⁶. However, this mean molecular weight was only achieved for the recombinant Bacillus strain where a gene coding for the hyaluronan synthase gene from Streptococcus equisimilis and the gene coding for the UDP-glucose dehydrogenase from Bacillus subtilis were integrated into the Bacillus subtilis genome under the control of the amyQ promoter, where at the same time the Bacillus subtilis-endogenous cxpY gene (which codes for a cytochrome P450 oxidase) was inactivated.

WO 05 012529 describes the preparation of transgenic tobacco plants which were transformed using nucleic acid molecules encoding for hyaluronan synthases from Chlorella-infecting viruses. In WO 05 012529, use was made, on the one hand, of nucleic acid sequences encoding for hyaluronan synthase of the Chlorella virus strain CVHI1 and, on the other hand, of the Chlorella virus strain CVKA1 for transforming tobacco plants. The synthesis of hyaluronan could only be demonstrated for a plant transformed with a nucleic acid encoding for a hyaluronan synthase isolated from the Chlorella virus strain CVKA1. For tobacco plants transformed with a nucleic acid sequence encoding for a hyaluronan synthase isolated from the Chlorella virus strain CVHI1, it was not possible to detect hyaluronan synthesis in the corresponding transgenic plants. The amount of hyaluronan synthesized by the only hyaluronan-producing transgenic tobacco plant in WO 05 012529 is stated as being about 4.2 μg of hyaluronan per ml of measured volume which, taking into account the description for carrying out the experiment in question, corresponds approximately to an amount of at most 12 μg of hyaluronan produced per gram of fresh weight of plant material.

Hyaluronan synthase catalyzes the synthesis of hyaluronan from the starting materials UDP-N-acetyl-glucosamine and UDP-glucuronic acid. Both starting materials mentioned are present in plant cells.

In plant cells, UDP-glucuronic acid serves as a metabolite for one of several possible synthesis paths of ascorbic acid (Lorence et al., 2004, Plant Physiol 134, 1200-1205) and as a central metabolite for the synthesis of the cell wall components pectin and hemicellulose which are synthesized in the endoplasmatic reticulum of the plant cell (Reiter, 1998, Plant Physiol Biochem 36(1), 167-176). The most important and the most frequently encountered monomer of pectin is D-galacturonic acid (2004, H. W. Heldt in “Plant Biochemistry”, 3rd Edition, Academic Press, ISBN 0120883910), which is synthesized using UDP-glucuronic acid. Furthermore, it is also possible, inter alia, to synthesize UDP-xylose, UDP-arabinose, UDP-galaturonic acid and UDP-apiose, metabolites for the synthesis of hemicellulose and pectin, using UDP-glucuronic acid (Seitz et al., 2000, Plant Journal, 21(6), 537-546). In plant cells, UDP-glucuronic acid can be synthesized either via the hexose phosphate metabolism comprising, inter alia, the conversion of UDP-glucose into UDP-glucuronic acid by UDP-Glc-DH or by the oxidative myo-inositol metabolic path comprising the conversion of glucuronate 1-phosphate to UDP-glucuronic acid by glucuronate 1-phosphate uridilyl transferase. The two metabolic paths for the synthesis of glucuronic acid appear to exist independently of one another, or alternatively in different tissues/development stages of Arabidopsis plants (Seitz et al., 2000, Plant Journal 21(6), 537-546). The respective contributions of the two metabolic paths mentioned (hexose phosphate and oxidative myo-inositol metabolic path) with respect to the synthesis of UDP-glucuronic acid have hitherto not been elucidated (Kärkönen, 2005, Plant Biosystems 139(1), 46-49).

The enyzme UDP-Glc-DH catalyzes the conversion of UDP-glucose into UDP-glucuronic acid. Samac et al. (2004, Applied Biochemistry and Biotechnology 113-116, Humana Press, Editor Ashok Mulehandani, 1167-1182) describe the tissue-specific overexpression of a UDP-Glc-DH from soybeans in phloem cells of Alfalfa with the aim of increasing the pectin content in the stems of these plants. Compared to the corresponding wild-type plants, the activity of the UDP-Glc-DH could be increased by more than 200%; however, the amount of pectin produced by the corresponding plants was lower than the amount of pectin produced by the corresponding wild-type plants. The amount of xylose and rhamnose monomers in the cell wall fraction of the transgenic plants in question was increased, whereas the amount of mannose monomers in the cell wall fraction was reduced.

The constitutive overexpression of a UDP-Glc-DH in Arabidosis plants resulted in the plants in question exhibiting aberrant growth compared to the corresponding wild-type plants and having a dwarf phenotype. The cell wall fraction of the corresponding plants had an increased amount of mannose and galactose and a reduced amount of xylose, arabinose and uronic acids compared to the corresponding wild-type plants (Roman, 2004, “Studies on The Role of UDP-Glc-DH in Polysaccharide Biosynthesis”, Dissertation, Acta Universitatis Upsaliensis, ISBN 91-554-6088-7, ISSN 0282-7476). Thus, these results contradict at least partially the results of Samac et al. (2004, Applied Biochemistry and Biotechnology 113-116, Humana Press, Editor Ashok Mulehandani, 1167-1182) which had found a reduced amount of mannose and an increased amount of xylose in the cell wall fraction of corresponding transgenic plants.

The production of hyaluronan by fermentation of bacteria strains is associated with high costs, since the bacteria have to be fermented in sealed sterile containers under expensive controlled culture conditions (see, for example, U.S. Pat. No. 4,897,349). Furthermore, the amount of hyaluronan which can be produced by fermentation of bacteria strains is limited by the production facilities present in each case. Here, it also has to be taken into account that fermenters, as a consequence of physical laws, cannot be built for excessively large culture volumes. Particular mention may be made here of homogeneous mixing of the substances fed in from the outside (for example essential nutrient sources for bacteria, reagents for regulating the pH, oxygen) with the culture medium required for efficient production, which, in large fermenters, can be ensured only with great technical expenditure, if at all.

The purification of hyaluronan from animal organisms is complicated owing to the presence, in animal tissues, of other mucopolysaccharides and proteins which specifically bind to hyaluronan. In patients, the use of hyaluronan-containing medicinal preparations contaminated by animal proteins can result in unwanted immunological reactions of the body (U.S. Pat. No. 4,141,973), in particular if the patient is allergic to animal proteins (for example chicken egg white). Furthermore, the amounts (yields) of hyaluronan which can be obtained from animal tissues in satisfactory quality and purity are low (cockscomb: 0.079% w/w, EP 0144019, U.S. Pat. No. 4,782,046), which necessitates the processing of large amounts of animal tissues. A further problem in the isolation of hyaluronan from animal tissues consists in effect that the molecular weight of hyaluronan during purification is reduced since animal tissues also contain a hyaluronan-degrading enzyme (hyaluronidase).

In addition to the hyaluronidases and exotoxins mentioned, Streptococcus strains also produce endotoxins which, when present in pharmacological products, pose risks for the health of the patient. In a scientific study, it was shown that even hyaluronan-containing medicinal products on the market contain detectable amounts of bacterial endotoxins (Dick et al., 2003, Eur J Opthalmol. 13(2), 176-184). A further disadvantage of the hyaluronan produced with the aid of Streptococcus strains is the fact that the isolated hyaluronan has a lower molecular weight than hyaluronan isolated from cockscombs (Lapcik et al. 1998, Chemical Reviews 98(8), 2663-2684). US 20030134393 describes the use of a Streptococcus strain for producing hyaluronan which synthesizes a particularly pronounced hyaluronan capsule (supercapsulated). The hyaluronan isolated after fermentation had a molecular weight of 9.1×10⁶. However, the yield was only 350 mg per liter.

Some of the disadvantages of producing hyaluronan by bacterial fermentation or by isolation from animal tissues can be avoided by producing hyaluronan using transgenic plants; however, the currently achieved amounts of hyaluronan which can be produced using transgenic plants would require a relatively large area under cultivation to produce relatively large amounts of hyaluronan. Furthermore, the isolation or purification of hyaluronan from plants having a lower hyaluronan content is considerably more complicated and costly than the isolation or purification from plants having a higher hyaluronan content.

Although hyaluronan has unusual properties, it is, owing to its scarcity and the high price, rarely, if at all, used for industrial applications.

Accordingly, it is an object of the present invention to provide means and methods which permit the provision of hyaluronan in sufficient amounts and quality and which make it possible to provide hyaluronan even for industrial applications and applications in the field of food and feed.

This object is achieved by the embodiments outlined in the claims.

Thus, the present invention relates to genetically modified plant cells or genetically modified plants having a nucleic acid molecule, stably integrated into their genome, encoding for a hyaluronan synthase, characterized in that said plant cells or said plants additionally have increased activity of a protein having an (enzymatic) UDP-glucose dehydrogenase (UDP-Glc-DH) activity compared to corresponding not genetically modified wild-type plant cells or not genetically modified wild-type plants.

Here, the genetic modification of genetically modified plant cells according to the invention or genetically modified plants according to the invention can be any genetic modification resulting in a stable integration of a nucleic acid molecule encoding for a hyaluronan synthase into a plant cell or a plant and increasing the activity of a protein having the (enzymatic) activity of a UDP-Glc-DH in genetically modified plant cells or genetically modified plants, compared to corresponding not genetically modified wild-type plant cells or not genetically modified wild-type plants.

In the context of the present invention, the term “wild-type plant cell” is to be understood as meaning plant cells which served as starting material for the preparation of the genetically modified plant cells according to the invention, i.e. their genetic information, apart from the genetic modifications introduced and resulting in a stable integration of a nucleic acid molecule encoding for a hyaluronan synthase and increasing the activity of a protein having the activity of a UDP-Glc-DH, corresponds to that of a genetically modified plant cell according to the invention.

In the context of the present invention, the term “wild-type plant” is to be understood as meaning plants which served as starting material for the preparation of the genetically modified plants according to the invention, i.e. their genetic information, apart from the genetic modifications introduced and resulting in a stable integration of a nucleic acid molecule encoding for a hyaluronan synthase and increasing the activity of a protein having the activity of a UDP-Glc-DH, corresponds to that of a genetically modified plant according to the invention.

In the context of the present invention, the term “corresponding” means that, when a plurality of objects are compared, the objects in question which are compared to one another have been kept under the same conditions. In the context of the present invention, the term “corresponding” in the context of wild-type plant cells or wild-type plants means that the plant cells or plants compared to one another were cultivated under the same cultivation conditions and that they have the same (culture) age.

In the context of the present invention, the term “hyaluronan synthase” (EC 2.4.1.212) is to be understood as meaning a protein which synthesizes hyaluronan from the substrates UDP-glucuronic acid (UDP-GlcA) and N-acetyl-glucosamine (UDP-GlcNAc). The hyaluronan synthesis is catalyzed according to the reaction schemes below:

nUDP-GlcA+nUDP-GlcNAc→beta-1,4-[GlcA-beta-1,3-GlcNAc]ₙ+2 nUDP

Nucleic acid molecules and corresponding protein sequences coding for hyaluronan synthases have been described, inter alia, for the following organisms: rabbit (Oryctolagus cuniculus) ocHas2 (EMBL AB055978.1, US 20030235893), ocHas3 (EMBL AB055979.1, US 20030235893); baboon (Papio anubis) paHas1 (EMBL AY463695.1); frog (Xenopus laevis) xlHas1 (EMBL M22249.1, US 20030235893), xlHas2 (DG42) (EMBL AF168465.1), xlHas3 (EMBL AY302252.1); human (Homo sapiens) hsHAS1 (EMBL D84424.1, US 20030235893), hsHAS2 (EMBL U54804.1, US 20030235893), hsHAS3 (EMBL AF232772.1, US 20030235893); mouse (Mus musculus), mmHas1 (EMBL D82964.1, US 20030235893), mmHAS2 (EMBL U52524.2, US 20030235893), mmHas3 (EMBL U86408.2, US 20030235893); cattle (Bos taurus) btHas2 (EMBL AJ004951.1, US 20030235893); chicken (Gallus gallus) ggHas2 (EMBL AF106940.1, US 20030235893); rat (Rattus norvegicus) rnHas1 (EMBL AB097568.1, Itano et al., 2004, J. Biol. Chem. 279(18) 18679-18678), rnHas2 (EMBL AF008201.1); mHas 3 (NCBI NM_{—}172319.1, Itano et al., 2004, J. Biol. Chem. 279(18) 18679-18678), horse (Equus caballus) ecHAS2 (EMBL AY056582.1, GI:23428486), pig (Sus scrofa) sscHAS2 (NCBI NM_{—}214053.1, GI:47522921), sscHas 3 (EMBLAB159675), zebra fish (Danio rerio) brHas1 (EMBL AY437407), brHas2 (EMBL AF190742.1) brHas3 (EMBL AF190743.1); Pasteurella multocida pmHas (EMBL AF036004.2); Streptococcus pyogenes spHas (EMBL, L20853.1, L21187.1, U.S. Pat. No. 6,455,304, US 20030235893); Streptococcus equis seHas (EMBL AF347022.1, AY173078.1), Streptococcus uberis suHasA (EMBL AJ242946.2, US 20030235893), Streptococcus equisimilis seqHas (EMBL AF023876.1, US 20030235893); Sulfolobus solfataricus ssHAS (US 20030235893), Sulfolobus tokodaii stHas (AP000988.1), Paramecium bursaria Chlorella Virus 1, cvHAS (EMBL U42580.3, PB42580, US 20030235893).

In the context of the present invention, the term “UDP-glucose dehydrogenase (UDP-Glc-DH)” (E.C. 1.1.1.22) is to be understood as meaning a protein which synthesizes, from UDP-glucose (UDP-Glc) and NAD⁺ UDP-glucaronic acid (UDP-GlcA) and NADH. This catalysis proceeds according to the following reaction scheme:

UDP-Glc+2 NAD⁺→UDP-GlcA+2 NADH

In the context of the present invention, the term “increased activity of a protein having the (enzymatic) activity of a UDP-Glc-DH” means an increased impression of endogenous genes coding for proteins having the activity of a UDP-Glc-DH and/or an increased amount of transcripts coding for proteins having the activity of a UDP-Glc-DH and/or an increased amount of protein having the activity of a UDP-Glc-DH in the cells and/or an increased enzymatic activity of proteins having the activity of a UDP-Glc-DH in the cells.

An increased expression can be determined, for example, by measuring the amount of transcripts coding for a protein having the activity of a UDP-Glc-DH, for example by Northern blot analysis or RT-PCR. Here, an increase preferably means an increase in the amount of transcripts compared to corresponding not genetically modified wild-type plant cells or not genetically modified wild-type plants by at least 50%, in particular by at least 70%, preferably by at least 85% and particularly preferably by at least 100%. An increase of the amount of transcripts coding for a protein having the activity of a UDP-Glc-DH also means that plants or plant cells having no detectable amounts of transcripts coding for a protein having the activity of a UDP-Glc-DH have, after genetic modification according to the invention, detectable amounts of transcripts coding for a protein having the activity of a UDP-Glc-DH.

An increase in the amount of protein having the activity of a UDP-Glc-DH resulting in an increased activity of these proteins in the plant cells in question can be determined, for example, by immunological methods, such as Western blot analysis, ELISA (Enzyme Linked Immuno Sorbent Assay) or RIA (Radio Immune Assay). Methods for preparing antibodies reacting specifically with a particular protein, i.e. bindings specifically to said protein, are known to the person skilled in the art (see, for example, Lottspeich and Zorbas (Eds.), 1998, Bioanalytik [Bioanalysis], Spektrum akad. Verlag, Heidelberg, Berlin, ISBN 3-8274-0041-4). Some companies (for example Eurogentec, Belgium) offer the preparation of such antibodies as an order service. Here, an increase in the amount of protein preferably means an increase in the amount of protein having an activity of a UDP-Glc-DH compared to corresponding not genetically modified wild-type plant cells or not genetically modified wild-type plants by at least 50%, in particular by at least 70%, preferably by at least 85% and particularly preferably by at least 100%. An increase in the amount of protein having an activity of a UDP-Glc-DH also means that plants or plant cells having no detectable amount of a protein having the activity of a UDP-Glc-DH have, after genetic modification according to the invention, a detectable amount of a protein having the activity of a UDP-Glc-DH.

The increased activity of a protein having the activity of a UDP-Glc-DH in plant extracts can be described by methods known to the person skilled in the art as described, for example, in WO 00 11192. A preferred method for determining the amount of the activity of a protein having the activity of a UDP-Glc-DH is given in General Methods, item 5.

An increased amount of (enzymatic) activity of proteins having the activity of a UDP-Glc-DH preferably means an increase of the activity of such proteins by at least 50%, preferably at least 70%, especially preferably by at least 85% and particularly preferably by at least 100% compared to corresponding not genetically modified wild-type plant cells or not genetically modified wild-type plants. An increase in the amount of (enzymatic) activity of proteins having the activity of a UDP-Glc-DH also means that plants or plant cells having no detectable amount of a protein having the activity of a UDP-Glc-DH have, after genetic modification according to the invention, a detectable amount of a protein having the activity of a UDP-Glc-DH.

In the context of the present invention, the term “genome” is to be understood as meaning the entire genetic material present in a plant cell. It is known to the person skilled in the art that, in addition to the nucleus, other compartments (for example plastids, mitochondria) also contain genetic material.

In the context of the present invention, the term “stably integrated nucleic acid molecule” is to be understood as meaning the integration of a nucleic acid molecule into the genome of the plant. A stably integrated nucleic acid molecule is characterized in that, during the replication of the corresponding integration site, it is multiplied together with the nucleic acid sequences of the host which border on the integration site, so that the integration site in the replicated DNA strand is surrounded by the same nucleic acid sequences as on the read strand which serves as a matrix for the replication.

A large number of techniques for stably integrating nucleic acid molecules into a plant host cell is available. These techniques include the transformation of plant cells with t-DNA using Agrobacterium tumefaciens or Agrobacterium rhizogenes as means of transformation, protoplast fusion, injection, electroporation of DNA, introduction of DNA by the biolistic approach and also further options (review in “Transgenic Plants”, Leandro ed., Humana Press 2004, ISBN 1-59259-827-7).

The use of agrobacterium-mediated transformation of plant cells has been subject to in-depth studies and has been described exhaustively in EP 120516; Hoekema, I N: The Binary Plant Vector System Offsetdrukkerij Kanters B. V. Alblasserdam (1985), Chapter V; Fraley et al., Crit. Rev. Plant Sci. 4, 1-46 and in An et al. EMBO J. 4, (1985), 277-287. For the transformation of potatoes see, for example, Rocha-Sosa et al., EMBO J. 8, (1989), 29-33), for the transformation of tomato plants see, for example, U.S. Pat. No. 5,565,347.

The transformation of monocotyledonous plants using vectors based on Agrobacterium transformation has been described, too (Chan et al., Plant Mol. Biol. 22, (1993), 491-506; Hiei et al., Plant J. 6, (1994) 271-282; Deng et al, Science in China 33, (1990), 28-34; Wilmink et al., Plant Cell Reports 11, (1992), 76-80; May et al., Bio/Technology 13, (1995), 486-492; Conner and Domisse, Int. J. Plant Sci. 153 (1992), 550-555; Ritchie et al, Transgenic Res. 2, (1993), 252-265). An alternative system for transforming monocotyledonous plants is the transformation using the biolistic approach (Wan and Lemaux, Plant Physiol. 104, (1994), 37-48; Vasil et al., Bio/Technology 11 (1993), 1553-1558; Ritala et al., Plant Mol. Biol. 24, (1994), 317-325; Spencer et al., Theor. Appl. Genet. 79, (1990), 625-631), the protoplast transformation, the electroporation of partially permeabilized cells, the introduction of DNA using glass fibers. In particular the transformation of corn has been described several times in the literature (cf., for example, WO95/06128, EP0513849, EP0465875, EP0292435; Fromm et al., Biotechnology 8, (1990), 833-844; Gordon-Kamm et al., Plant Cell 2, (1990), 603-618; Koziel et al., Biotechnology 11 (1993), 194-200; Moroc et al., Theor. AppI. Genet. 80, (1990), 721-726). The transformation of other grasses, such as, for example, switchgrass (Panicum virgatum) has also been described (Richards et al., 2001, Plant Cell Reporters 20, 48-54).

The successful transformation of other cereal species has also been described, for example for barley (Wan and Lemaux, s.o.; Ritala et al., s.o.; Krens et al., Nature 296, (1982), 72-74) and for wheat (Nehra et al., Plant J. 5, (1994), 285-297; Becker et al., 1994, Plant Journal 5, 299-307). All of the above methods are suitable in the context of the present invention.

Compared to the prior art, genetically modified plant cells according to the invention or genetically modified plants according to the invention offer the advantage that they produce higher amounts of hyaluronan than plants having only the activity of a hyaluronan synthase. This allows hyaluronan to be produced at little expense since the isolation of hyaluronan from plants having a higher hyaluronan content is less complicated and more cost efficient. Furthermore, compared to the plants described in the prior art, smaller cultivation areas are required to produce hyaluronan using the genetically modified plants according to the invention. This leads to the possibility to provide hyaluronan in sufficient amounts even for industrial application where it is currently not used owing to its scarcity and the high price. Virus-infected plant organisms of the genus Chlorella are unsuitable for producing relatively large amounts of hyaluronan. In the production of hyaluronan, virus-infected algae have the disadvantage that the genes required for hyaluronan synthesis are not stably integrated into their genome (Van Etten and Meints, 1999, Annu. Rev. Microbiol. 53, 447-494), so that, for producing hyaluronan, the virus infection has to be repeated. Accordingly, it is not possible to isolate individual Chlorella cells which synthesize continuously the desired quality and quantity of hyaluronan. Furthermore, in virus-infected Chlorella algae, hyaluronan is only produced for a limited period of time, and as a result of the lysis caused by the virus, the algae are killed only about 8 hours after the infection (Van Etten et al., 2002, Arch Virol 147, 1479-1516). In contrast, the present invention offers the advantage that the genetically modified plant cells according to the invention and the genetically modified plants according to the invention can be propagated in an unlimited manner vegetatively or sexually and that they produce hyaluronan continuously. The transgenic plants described in WO 05 012529, which have a nucleic acid molecule coding for a hyaluronan synthase, synthesize a relatively small amount of hyaluronan. In contrast, the present invention offers the advantage that genetically modified plant cells according to the invention and genetically modified plants according to the invention synthesize considerably higher amounts of hyaluronan.

Accordingly, the present invention also provides genetically modified plant cells according to the invention or genetically modified plants according to the invention which synthesize hyaluronan.

It has been observed that, over the development time, hyaluronan accumulates in plant tissue; accordingly, the amount of hyaluronan with respect to the fresh weight or with respect to the dry weight in the genetically modified plant cells according to the invention or in the genetically modified plants according to the invention is to be determined with particular preference during harvesting or (one or two) days before harvesting of the plant cells in question or the plants in question. Here, use is made in particular of plant material (for example tubers, seeds, leaves) with respect to the amount of hyaluronan which is to be used for further processing.

Genetically modified plant cells according to the invention or genetically modified plants according to the invention which synthesize hyaluronan can be identified by isolating the hyaluronan that is synthesized by them and proving its structure.

Since plant tissue has the advantage that it does not contain hyaluronidases, a simple and rapid isolation method can be used for confirming the presence of hyaluronan in genetically modified plant cells according to the invention or genetically modified plants according to the invention. To this end, water is added to the plant tissue to be examined and the plant tissue is then comminuted mechanically (with the aid of, for example, a bead mill, a beater mill, a Warring blender, a juice extractor, etc.). If required, more water may then be added to the suspension, and cell debris and water-insoluble components are then removed by centrifugation or sieving. The presence of hyaluronan in the supernatant obtained after centrifugation can then be demonstrated using, for example, a protein which binds specifically to hyaluronan. A method for detecting hyaluronan with the aid of a protein that binds specifically to hyaluronan is described, for example, in U.S. Pat. No. 5,019,498. Test kits for carrying out the method described in U.S. Pat. No. 5,019,498 are commercially available (for example the hyaluronic acid (HA) test kit from Corgenix, Inc., Colorado, USA, Prod. No. 029-001); see also General Methods item 4). In parallel, it is possible to initially digest an aliquot of the centrifugation supernatant obtained with a hyaluronidase and then to confirm the presence of hyaluronan with the aid of a protein that specifically binds to hyaluronan, as described above. By the action of the hyaluronidase in the parallel batch, the hyaluronan present therein is degraded, so that after complete digestion it is no longer possible to detect significant amounts of hyaluronan.

The presence of hyaluronan in the centrifugation supernatant can furthermore also be confirmed using other analysis methods, such as, for example, IR, NMR or mass spectroscopy.

As already mentioned above, it has hitherto not been elucidated which metabolic path (hexose phosphate or oxidative myoinositol metabolic path) is the main path used for synthesizing UDP-glucuronic acid in plant cells, and whether both metabolic paths make a different quantitative contribution to the synthesis of UDP-glucuronic acid depending on the tissue and/or development stage of the plant. Furthermore, the overexpression of a UDP-Glc-DH in transgenic plants did not give consistent results, and the aim of increasing the pectin content of the cell wall using such an approach could not be achieved. In addition, the regulation of the activity of proteins using the activity of a UDP-Glc-DH is inhibited by UDP-xylose. This was demonstrated for the proteins in question originating from procaryonts (Campbell et al., 1997, J. Biol. Chem. 272(6), 3416-3422; Schiller et al., 1973, Biochim. Biophys Acta 293(1), 1-10), from animal organisms (Balduini et al., 1970, Biochem. J. 120(4), 719-724) and from plants (Hinterberg, 2002, Plant Physiol. Biochem. 40, 1011-1017).

There are no indications in the literature what may limit the amount of hyaluronan synthesized in plant cells.

Accordingly, it has surprisingly been found that genetically modified plant cells or genetically modified plants having a nucleic acid molecule coding for a hyaluronan synthase and having additionally increased UDP-Glc-DH activity compared to genetically modified plant cells or genetically modified plants having (only) hyaluronan synthase activity produce significantly high amounts of hyaluronan.

In a preferred embodiment, the present invention relates to genetically modified plant cells according to the invention or genetically modified plants according to the invention, characterized in that they produce an increased amount of hyaluronan compared to genetically modified plant cells or compared to genetically modified plants which (only) have the activity of a hyaluronan synthase or compared to genetically modified plant cells or compared to genetically modified plants having the activity of a hyaluronan synthase and no increased activity of a protein having the activity of a UDP-Glc-DH,

In the context of the present invention, the term “plant cell or plant (only) having the activity of a hyaluronan synthase” is to be understood as meaning a genetically modified plant cell or a genetically modified plant where the genetic modification consists in that it comprises a nucleic acid molecule coding for a hyaluronan synthase, compared to corresponding not genetically modified wild-type plant cells or not genetically modified wild-type plants.

In particular, “plant cells or plants (only) having the activity of a hyaluronan synthase” are characterized in that they synthesize hyaluronan and that they have no additional genetic modifications other than the introduction of a nucleic acid molecule coding for a hyaluronan synthase into not genetically modified wild-type plant cells or not genetically modified wild-type plants. Preferably, such plants do not have an increased activity of a protein having the activity of a UDP-Glc-DH.

The amount of hyaluronan produced by plant cells or plants can be determined with the aid of the methods which have already been described above, for example using a commercial test kit (for example the hyaluronic acid (HA) test kit from Corgenix, Inc., Colorado, USA, Prod. No. 029-001). A method which is preferred in the context of the present invention for determining the hyaluronan content in plant cells or plants is described under General Methods, item 4.

In a further embodiment of the present invention, the genetically modified plant cells according to the invention or the genetically modified plants according to the invention are plant cells of a green terrestrial plant or green terrestrial plants, respectively, which synthesize hyaluronan.

In the context of the present invention, the term “green terrestrial plant (Embryophyta)” is to be understood as defined in Strasburger, “Lehrbuch der Botanik” [Textbook of Botany], 34th ed., Spektrum Akad. Veri., 1999, (ISBN 3-8274-0779-6).

A preferred embodiment of the present invention relates to genetically modified plant cells according to the invention of multicellular plants or genetically modified plants according to the invention which are multicellular organisms. Accordingly, this embodiment relates to plant cells or plants which do not originate from single-cell plants (protists) or which are not protists.

The genetically modified plant cells according to the invention or the genetically modified plants according to the invention may, in principle, be plant cells and plants, respectively, of any plant species, i.e. both monocotyledonous and dicotyledonous plants. They are preferably crop plants, i.e. plants cultivated by man for the purpose of feeding man and animal or for producing biomass and/or for preparing substances for technical, industrial purposes (for example corn, rice, wheat, alfalfa, rye, oats, barley, manioc, potato, tomato, switchgrass (Panicum virgatum), sago, mung beans, pas, sorghum, carrots, aubergine, radish, oilseed rape, soybeans, peanuts, cucumbers, pumpkins, melons, leek, garlic, cabbage, spinach, sweet potato, asparagus, courgettes, lettuce, artichokes, sweetcom, parsnip, scorzonera, Jerusalem artichoke, banana, sugarbeet, sugarcane, beetroot, broccoli, cabbage, onion, yellow beet, dandelion, strawberry, apple, apricot, plum, peach, grapevines, cauliflower, celery, bell peppers, swede, rhubarb). Particularly preferred are tomato or potato plants.

In a preferred embodiment, the present invention relates to genetically modified plant cells according to the invention or genetically modified plants according to the invention where the nucleic acid molecule coding for hyaluronan synthase is characterized in that it codes for a viral hyaluronan synthase. The nucleic acid molecule coding for the hyaluronan synthase preferably codes for a hyaluronan synthase of a virus which infects algae.

With respect to an algae-infecting virus, the nucleic acid molecule which codes for a hyaluronan synthase preferably codes for a hyaluronan synthase of a Chlorella-infecting virus, particularly preferably a hyaluronan synthase of a Paramecium bursaria Chlorella Virus 1 and especially preferably a hyaluronan synthase of a Paramecium bursaria Chlorella virus of an H1 strain.

In a further preferred embodiment, the present invention relates to genetically modified plant cells according to the invention or genetically modified plants according to the invention where the nucleic acid molecule which codes for the hyaluronan synthase is characterized in that the codons of the nucleic acid molecule coding for a hyaluronan synthase are modified compared to the codons of the nucleic acid molecule coding for the hyaluronan synthase of the organism that the hyaluronan synthase originates from. With particular preference, the codons of the hyaluronan synthase have been modified such that they are adapted to the frequency of the use of the codons of the plant cell or the plant into whose genome they are integrated or to be integrated.

Owing to the degeneration of the genetic code, amino acids can be encoded by one or more codons. In different organisms, the codons coding for an amino acid are used at different frequencies. Adapting the codon of a coding nucleic acid sequence to the frequency of their use in the plant cell or in the plant into whose genome the sequence to be expressed is to be integrated may contribute to an increased amount of translated protein and/or to the stability of the mRNA in question in the particular plant cells or plants. The frequency of use of codons in the plant cells or plants in question can be determined by the person skilled in the art by examining as many coding nucleic acid sequences of the organism in question as possible for the frequency with which certain codons are used for coding a certain amino acid. The frequency of the use of codons of certain organisms is known to the person skilled in the art and can be determined in a simple and rapid manner using computer programs. Suitable computer programs are publicly accessible and provided for free inter alia on the internet (for example http://gcua.schoedl.de/; http://www.kazusa.or.jp/codon/; http://www.entelechon.com/eng/cutanalysis.html).

Adapting the codons of a coding nucleic acid sequence to the frequency of their use in the plant cell or in the plant into whose genome the sequence to be expressed is to be integrated can be carried out by in vitro mutagenesis or, preferably, by de novo synthesis of the gene sequence. Methods for the de novo synthesis of nucleic acid sequences are known to the person skilled in the art. A de novo synthesis can be carried out, for example, by initially synthesizing individual nucleic acid oligonucleotides, hybridizing these with oligonucleotides complementary thereto, so that they form a DNA double strand, and then ligating the individual double-stranded oligonucleotides such that the desired nucleic acid sequence is obtained. The de novo synthesis of nucleic acid sequences including the adaptation of the frequency with which the codons are used to a certain target organism can also be sourced out to companies offering this service (for example Entelechon GmbH, Regensburg, Germany).

The nucleic acid molecule coding for the hyaluronan synthase is preferably characterized in that it codes for a hyaluronan synthase whose amino acid sequence is at least 70%, preferably at least 80%, with preference at least 90% and especially preferably at least 95% identical to the amino acid sequence shown under SEQ ID NO 2. In a particularly preferred embodiment, the nucleic acid molecule coding for the hyaluronan synthase is characterized in that it codes for a hyaluronan synthase having the amino acid sequence shown under SEQ ID No 2.

In a further embodiment, the nucleic acid molecule coding for a hyaluronan synthase is at least 70%, preferably at least 80%, with preference at least 90% and especially preferably at least 95% identical to the nucleic acid sequence shown under SEQ ID NO 1 or SEQ ID NO 3. In a particularly preferred embodiment, the nucleic acid molecule coding for the hyaluronan synthase is characterized in that it has the nucleic acid sequence shown under SEQ ID NO 3.

On Aug. 25, 2004, the plasmid IC 341-222, comprising a synthetic nucleic acid molecule coding for a Paramecium bursaria Chlorella virus hyaluronan synthase was deposited at the Deutsche Sammlung von Mikroorganismen und Zellkulturen GmbH, Mascheroder Weg 1b, 38124 Brunswick, Germany, under the number DSM16664, in accordance with the Budapest treaty. The amino acid sequence shown in SEQ ID NO 2 can be derived from the coding region of the nucleic acid sequence integrated into the plasmid IC 341-222 and codes for a Paramecium bursaria Chlorella virus hyaluronan synthase.

Accordingly, the present invention also relates to genetically modified plant cells according to the invention or genetically modified plants according to the invention where the nucleic acid molecule which codes for the hyaluronan synthase is characterized in that it codes for a protein whose amino acid sequence can be derived from the coding region of the nucleic acid sequence inserted into plasmid DSM16664 or that it codes for a protein whose amino acid sequence is at least 70%, preferably at least 80%, with preference at least 90% and especially preferably at least 95% identical to the amino acid sequence which can be derived from the coding region of the nucleic acid sequence inserted into plasmid DSM16664.

The present invention also relates to genetically modified plant cells according to the invention or genetically modified plants according to the invention where the nucleic acid molecule coding for hyaluronan synthase is characterized in that it is the hyaluronan-synthase-encoding nucleic acid sequence integrated into plasmid DSM16664 or that it is at least 70%, preferably at least 80%, with preference at least 90% and especially preferably at least 95% identical to the nucleic acid sequence integrated into plasmid DSM16664.

The present invention furthermore relates to genetically modified plant cells according to the invention or genetically modified plants according to the invention which are characterized in that they have a foreign nucleic acid molecule stably integrated into their genome, said foreign nucleic acid molecule increasing the activity of a protein having the activity of a UDP-Glc-DH compared to corresponding not genetically modified wild-type plant cells or corresponding not genetically modified wild-type plants.

In the context of the present invention, the term “foreign nucleic acid molecule” is to be understood as meaning a molecule which either does not naturally occur in the corresponding wild-type plant cells or which does not naturally occur in the concrete spatial arrangement in wild-type plant cells or which is localized at a site in the genome of the wild-type plant cell where it does not naturally occur. Preferably, the foreign nucleic acid molecule is a recombinant molecule comprising various elements whose combination or specific spatial arrangement does not naturally occur in plant cells.

In the context of the present invention, the term “recombinant nucleic acid molecule” is to be understood as meaning a nucleic acid molecule which comprises various nucleic acid molecules which are not naturally present in a combination like that present in a recombinant nucleic acid molecule. Thus, recombinant nucleic acid molecules may, in addition to nucleic acid molecules coding for a hyaluronan synthase and/or a protein having the activity of a UDP-Glc-DH, additionally comprise nucleic acid sequences which are not naturally present in combination with the nucleic acid molecules mentioned. The additional nucleic acid sequences mentioned which are present on a recombinant nucleic acid molecule in combination with a nucleic acid molecule encoding for a hyaluronan synthase or a protein having the activity of a UDP-Glc-DH may be any sequences. For example, they may be genomic plant nucleic acid sequences. The additional nucleic acid sequences are preferably regulatory sequences (promoters, termination signals, enhancers), particularly preferably regulatory sequences which are active in plant tissue, especially preferably tissue-specific regulatory sequences which are active in plant tissue. Methods for generating recombinant nucleic acid molecules are known to the person skilled in the art and comprise genetic engineering methods, such as, for example, linking of nucleic acid molecules by ligation, genetic recombination or the de novo synthesis of nucleic acid molecules (see, for example, Sambrok et al., Molecular Cloning, A Laboratory Manual, 3rd edition (2001) Cold Spring Harbor Laboratory Press, Cold Spring Harbor, N.Y. ISBN: 0879695773, Ausubel et al., Short Protocols in Molecular Biology, John Wiley & Sons; 5th edition (2002), ISBN: 0471250929).

Genetically modified plant cells and genetically modified plants having a foreign nucleic acid molecule stably integrated into their genome or a plurality of foreign nucleic acid molecules stably integrated into their genome which code for hyaluronan synthase and which increase the activity of a protein having the activity of a UDP-Glc-DH compared to corresponding not genetically modified wild-type plant cells or not genetically modified wild-type plants can be distinguished from said wild-type plant cells and said wild-type plants, respectively, inter alia by the fact that they comprise a foreign nucleic acid molecule which does not naturally occur in wild-type plant cells and wild-type plants, respectively, or in that such a molecule is integrated at a site in the genome of the genetically modified plant cell according to the invention or in the genome of the genetically modified plant according to the invention where it does not occur in wild-type plant cells and wild-type plants, respectively, i.e. in a different genomic environment. Furthermore, such genetically modified plant cells according to the invention and genetically modified plants according to the invention can be distinguished from not genetically modified wild-type plant cells and not genetically modified wild-type plants, respectively, in that they comprise at least one copy of the foreign nucleic acid molecule stably integrated into their genome, if appropriate in addition to copies of such a molecule naturally present in the wild-type plant cells or wild-type plants. If the foreign nucleic acid molecule(s) introduced into the genetically modified plant cells according to the invention or the genetically modified plant according to the invention are additional copies of molecules already naturally present in the wild-type plant cells or the wild-type plants, the genetically modified plant cells according to the invention and the genetically modified plants according to the invention can be distinguished from wild-type plant cells and wild-type plants, respectively, in particular by the fact that this additional copy/these additional copies is/are localized at sites in the genome where it/they is/are not present in wild-type plant cells and wild-type plants, respectively.

The stable integration of a nucleic acid molecule into the genome of a plant cell or a plant can be demonstrated by genetic methods and/or methods of molecular biology. A stable integration of a nucleic acid molecule into the genome of a plant cell or the genome of a plant is characterized in that in the progeny which has inherited said nucleic acid molecule, the stably integrated nucleic acid molecule is present in the same genomic environment as in the parent generation. The presence of a stable integration of a nucleic acid sequence in the genome of a plant cell or in the genome of a plant can be demonstrated using methods known to the person skilled in the art, inter alia with the aid of Southern blot analysis of the RFLP analysis (Restriction Fragment Length Polymorphism) (Nam et al., 1989, The Plant Cell 1, 699-705; Leister and Dean, 1993, The Plant Journal 4 (4), 745-750), with methods based on PCR, such as, for example, the analysis of differences in length in the amplified fragment (Amplified Fragment Length Polymorphism, AFLP) (Castiglioni et al., 1998, Genetics 149, 2039-2056; Meksem et al., 2001, Molecular Genetics and Genomics 265, 207-214; Meyer et al., 1998, Molecular and General Genetics 259, 150-160) or using amplified fragments cleaved using restriction endonucleases (Cleaved Amplified Polymorphic Sequences, CAPS) (Konieczny and Ausubel, 1993, The Plant Journal 4, 403-410; Jarvis et al., 1994, Plant Molecular Biology 24, 685-687; Bachem et al., 1996, The Plant Journal 9 (5), 745-753).

In principle, the foreign nucleic acid molecule may be any nucleic acid molecule which increases, in the plant cell or plant, the activity of a protein having the activity of a UDP-Glc-DH.

In the context of the present invention, genetically modified plant cells according to the invention and genetically modified plants according to the invention can also be prepared by using insertion mutagenesis (review: Thorneycroft et al., 2001, Journal of experimental Botany 52 (361), 1593-1601). In the context of the present invention, insertion mutagenesis is to be understood as meaning in particular the insertion of transposons or transfer DNA (t-DNA) into a gene or into the vicinity of a gene coding for a protein having the activity of a UDP-Glc-DH, thus increasing the activity of a protein having the activity of a UDP-Glc-DH in the cell in question.

The transposons may either be transposons which occur naturally in the cell (endogenous transposons) or those which are not naturally present in said cell but were introduced into the cell by genetic engineering, such as, for example, transformation of the cell (heterologous transposons). The modification of the expression of genes by transposons is known to the person skilled in the art. A review of the use of endogenous and heterologous transposons as tools in plant biotechnology is given in Ramachandran and Sundaresan (2001, Plant Physiology and Biochemistry 39, 234-252).

t-DNA insertion mutagenesis is based on the fact that certain sections (t-DNA) of Ti plasmids from Agrobacterium can be integrated into the genome of plant cells. The site of integration into the plant chromosome is not fixed, integration can be in any location. If the t-DNA is integrated into a section or into the vicinity of a section of the chromosome representing a gene function, this may result in an increased gene expression and thus also a change in the activity of the protein encoded by the gene in question.

The sequences inserted into the genome (in particular transposons or t-DNA) are characterized in that they comprise sequences resulting in the activation of regulatory sequences of a gene coding for a protein having the activity of a UDP-Glc-DH (“activation tagging”). Preferably, the sequences inserted into the genome (in particular transposons or t-DNA) are characterized in that they are integrated into the vicinity of endogenous nucleic acid molecules in the genome of the plant cell or the plant coding for a protein having the activity of a UDP-Glc-DH.

Genetically modified plant cells according to the invention and genetically modified plants according to the invention can be generated, for example, using the method of activation tagging (see, for example, Walden et al., Plant J. (1991), 281-288; Walden et al., Plant Mol. Biol. 26 (1994), 1521-1528). This method is based on the activation of endogenous promoters by enhancer sequences, such as, for example, the enhancer of the 35S RNA promoter of the cauliflower mosaic virus or the octopine synthase enhancer.

In the context of the present invention, the term “t-DNA activation tagging” is to be understood as meaning a t-DNA fragment comprises enhancer sequences and, by integration into the genome of a plant cell, increases the activity of a protein having the activity of a UDP-Glc-DH.

In the context of the present invention, the term “transposon activation tagging” is to be understood as meaning a transposon which comprises enhancer sequences and, by integration into the genome of a plant cell, increases the activity of a protein having the activity of a UDP-Glc-DH.

A particularly preferred embodiment of the present invention relates to genetically modified plant cells according to the invention or genetically modified plants according to the invention which are characterized in that a foreign nucleic acid molecule codes for a protein having the enzymatic activity of a UDP-Glc-DH.

According to the invention, the foreign nucleic acid molecule coding for a protein having the enzymatic activity of a UDP-Glc-DH may originate from any organism; preferably, said nucleic acid molecule originates from bacteria, fungi, animals, plants or viruses, particularly preferably from bacteria, plants or viruses especially preferably from viruses. With respect to viruses, the foreign nucleic acid molecule coding for a protein having the enzymatic activity of a UDP-Glc-DH preferably originates from a virus which infects algae, with preference from a virus which infects algae of the genus Chlorella, particularly preferably from a Paramecium bursaria Chlorella virus and especially preferably from a Paramecium bursaria Chlorella virus of an H1 strain

Instead of the naturally occurring nucleic acid molecule coding for a protein having the enzymatic activity of a UDP-Glc-DH, it is also possible for a nucleic acid molecule generated by mutagenesis to be introduced into the genetically modified plant cells according to the invention or the genetically modified plants according to the invention, where said mutagenized foreign nucleic acid molecule is characterized in that it codes for a protein having the enzymatic activity of a UDP-Glc-DH with reduced inhibition by metabolites (for example of the glucoronic acid metabolism).

Nucleic acid molecules coding for a protein having the activity of a UDP-Glc-DH are described in the literature and known to the person skilled in the art. Thus, nucleic acid molecules coding for a protein having the activity of a UDP-Glc-DH are described from viruses, for example for the Chlorella virus 1 (NCBI acc No NC_{—}000852.3), from bacteria, for example for Escherichia coli (EMBL acc No: AF176356.1), from fungi, for example for Aspergillus niger (EMBL acc No AY594332.1), Cryptococcus neoformans (EMBL acc No AF405548.1), from insects for example for Drosophila melanogaster (EMBL acc No AF001310.1), from vertebrates for example for Homo sapiens (EMBL acc No AF061016.1), Mus musculus (EMBL acc No AF061017.1), Bos taurus (EMBL acc No AF095792.1), Xenopus laevis (EMBL acc No AY762616.1) or from plants for example for poplar (EMBL acc No AF053973.1), Colocasia esculenta (EMBL acc No AY222335.1), Dunaliella salina (EMBL acc No AY795899.1) Glycine max (EMBL acc No U53418.1).

In a preferred embodiment, the present invention relates to genetically modified plant cells according to the invention and genetically modified plants according to the invention where the foreign nucleic acid molecule coding for a protein having the activity of a UDP-Glc-DH is selected from the group consisting of
a) nucleic acid molecules coding for a protein having the amino acid sequence given under SEQ ID NO 5;
b) nucleic acid molecules coding for a protein whose sequence is at least 60% identical to the amino acid sequence given under SEQ ID NO 5;
c) nucleic acid molecules comprising the nucleotide sequence shown under SEQ ID NO 4 or a sequence complementary thereto or the nucleotide sequence shown under SEQ ID NO 6 or a sequence complementary thereto;
d) nucleic acid molecules which are at least 70% identical to the nucleic acid sequences described under a) or c);
e) nucleic acid molecules which hybridize under stringent conditions with at least one strand of the nucleic acid sequences described under a) or c);
f) nucleic acid molecules whose nucleotide sequence deviates from the sequence of the nucleic acid molecules mentioned under a) or c) owing to the degeneration of the genetic code; and
g) nucleic acid molecules which are fragments, allelic variants and/or derivatives of the nucleic acid molecules mentioned under a), b), c), d), e) or f).

In the context of the present invention, the term “hybridization” means a hybridization under conventional hybridization conditions, preferably under stringent conditions, as described, for example, in Sambrock et al., Molecular Cloning, A Laboratory Manual, 2 ed. (1989) Cold Spring Harbor Laboratory Press, Cold Spring Harbor, N.Y.). With particular preference, “hybridization” means a hybridization under the following conditions:

Hybridization buffer:

2×SSC; 10×Denhardt solution (Fikoll 400+PEG+BSA; ratio 1:1:1); 0.1% SDS; 5 mM

EDTA; 50 mM Na2HPO4; 250 μg/ml of herring sperm DNA; 50 μg/ml of tRNA; or

25 M sodium phosphate buffer pH 7.2; 1 mM EDTA; 7% SDS

Hybridization temperature:
T=65 to 68° C.
Wash buffer: 0.1×SSC; 0.1% SDS
Wash temperature: T=65 to 68° C.

Nucleic acid molecules which hybridize with nucleic acid molecules coding for a protein having the activity of a UDP-Glc-DH may originate from any organism; accordingly, they may originate from bacteria, fungi, animals, plants or viruses.

Nucleic acid molecules hybridizing with nucleic acid molecules coding for protein having the activity of a UDP-Glc-DH preferably originate from a virus infecting the algae, preferably from a virus infecting the algae of the genus Chlorella, particularly preferably from a Paramecium bursaria Chlorella virus and most preferably from an Hi strain of Paramecium bursaria Chlorella virus.

Nucleic acid molecules which hybridize with the molecules mentioned may be isolated, for example, from genomic or from cDNA libraries. Such nucleic acid molecules can be identified and isolated using the nucleic acid molecules mentioned or parts of these molecules or the reverse complements of these molecules, for example by hybridization according to standard methods (see, for example, Sambrook et al., 1989, Molecular Cloning, A Laboratory Manual, 2 ed. Cold Spring Harbor Laboratory Press, Cold Spring Harbor, N.Y.) or by amplification using PCR.

As hybridization sample for isolating a nucleic acid sequence coding for a protein having the activity of a UDP-Glc-DH, it is possible to use, for example, nucleic acid molecules having exactly or essentially the nucleotide sequence given under SEQ ID NO 4 or under SEQ ID NO 6, or parts of these sequences.

The fragments used as hybridization samples may also be synthetic fragments or oligonucleotides prepared using the customary synthesis techniques, whose sequence is essentially identical to the nucleic acid molecule described in the context of the present invention. Once genes which hybridize with the nucleic acid sequences described in the context of the present invention are identified and isolated, the sequence should be determined and the properties of the proteins coded for by this sequence should be analyzed to determine whether they are proteins having the activity of a UDP-Glc-DH. Methods of how to determine whether a protein has the activity of a protein having the activity of a UDP-Glc-DH (for example De Luca et al., 1976, Connective Tissue Research 4, 247-254; Bar-Peled et al., 2004, Biochem. J. 381, 131-136; Turner and Botha, 2002, Archives Biochem. Biophys. 407, 209-216) are known to the person skilled in the art and extensively described in the literature.

The molecules hybridizing with the nucleic acid molecules described in the context of the present invention comprise in particular fragments, derivatives and allelic variants of the nucleic acid molecules mentioned. In the context of the present invention, the term “derivative” means that the sequences of these molecules differ in one or more positions from the sequences of the nucleic acid molecules described above and are highly identical to these sequences. The differences to the nucleic acid molecules described above may, for example, be due to deletion, addition, substitution, insertion or recombination.

In the context of the present invention, the term “identity” means a sequence identity over the entire length of the coding region of a nucleic acid molecule or the entire length of an amino acid sequence coding for a protein of at least 60%, in particular in identity of at least 70%, preferably of at least 80%, particularly preferably of at least 90% and especially preferably of at least 95%. In the context of the present invention, the term “identity” is to be understood as meaning the number of identical amino acids/nucleotides (identity) with other proteins/nucleic acids, expressed in percent. Preferably, the identity with respect to a protein having the activity of a UDP-Glc-DH is determined by comparison with the amino acid sequence given under SEQ ID NO 5 and the identity with respect to a nucleic acid molecule coding for a protein having the activity of a UDP_Glc_DH is determined by comparison with the nucleic acid sequences given under SEQ ID NO 4 or SEQ ID NO 6 with other proteins/nucleic acids with the aid of computer programs. If sequences to be compared with one another are of different lengths, the identity is to be determined by determining the identity in percent of the number of amino acids which the shorter sequence shares with the longer sequence. Preferably, the identity is determined using the known and publicly available computer program ClustalW (Thompson et al., Nucleic Acids Research 22 (1994), 4673-4680). ClustalW is made publicly available by Julie Thompson (Thompson@EMBL-Heidelberg.DE) and Toby Gibson (Gibson@EMBL-Heidelberg.DE), European Molecular Biology Laboratory, Meyerhofstrasse 1, D 69117 Heidelberg, Germany. ClustalW can also be downloaded from various internet pages, inter alia from IGBMC (Institut de Génétique et de Biologie Moléculaire et Cellulaire, B. P. 163, 67404 Illkirch Cedex, France; ftp://ftp-igbmc.u-strasbg.fr/pub/) and from EBI (ftp://ftp.ebi.ac.uk/pub/software/) and all mirrored internet pages of the EBI (European Bioinformatics Institute, Wellcome Trust Genome Campus, Hinxton, Cambridge CB10 1SD, UK).

Preferably, use is made of the ClustalW computer program of version 1.8 to determine the identity between proteins described in the context of the present invention and other proteins. Here, the parameters have to be set as follows: KTUPLE=1, TOPDIAG=5, WINDOW=5, PAIRGAP=3, GAPOPEN=10, GAPEXTEND=0.05, GAPDIST=8, MAXDIV=40, MATRIX=GONNET, ENDGAPS(OFF), NOPGAP, NOHGAP.

Preferably, use is made of the ClustalW computer program of version 1.8 to determine the identity for example between the nucleotide sequence of the nucleic acid molecules described in the context of the present invention and the nucleotide sequence of other nucleic acid molecules. Here, the parameters have to be set as follows: KTUPLE=2, TOPDIAGS=4, PAIRGAP=5, DNAMATRIX:IUB, GAPOPEN=10, GAPEXT=5, MAXDIV=40, TRANSITIONS: unweighted.

Identity furthermore means that there is a functional and/or structural equivalence between the nucleic acid molecules in question or the proteins encoded by them. The nucleic acid molecules which are homologous to the molecules described above and represent derivatives of these molecules are generally variations of these molecules which represent modifications having the same biological function. They may be either naturally occurring variations, for example sequences from other species, or mutations, where these mutations may have occurred in a natural manner or were introduced by targeted mutagenesis. Furthermore, the variations may be synthetically produced sequences. The allelic variants may be either naturally occurring variants or synthetically produced variants or variants generated by recombinant DNA techniques. A special form of derivatives are, for example, nucleic acid molecules which differ from the nucleic acid molecules described in the context of the present invention owing to the degeneration of the genetic code.

The various derivatives of the nucleic acid molecules coding for a protein having the activity of a UDP-Glc-DH have certain common characteristics.

These may, for example, be biological activity, substrate specificity, molecular weight, immunological reactivity, conformation, etc., and also physical properties, such as, for example, the mobility properties in gel electrophoresis, chromatographic behavior, sedimentation coefficients, solubility, spectroscopic properties, stability, pH optimum, temperature optimum, etc. Preferred properties of proteins having the activity of a UDP-Glc-DH are known to the person skilled in the art, have already been mentioned above and are to apply here in an analogous manner.

In a further preferred embodiment, the present invention relates to genetically modified plant cells according to the invention or genetically modified plants according to the invention where nucleic acid molecules coding for a protein having the enzymatic activity of a UDP-Glc-DH are characterized in that the codons of said nucleic acid molecules are different from the codons of the nucleic acid molecules which code for said protein having the enzymatic activity of a UDP-Glc-DH of the parent organism. Particularly preferably, the codons of the nucleic acid molecules coding for a protein having the enzymatic activity of a UDP-Glc-DH are changed thus that they are adapted to the frequency of use of the codons of the plant cell or the plant into whose genome they are integrated or to be integrated.

The present invention furthermore provides genetically modified plant cells according to the invention or genetically modified plants according to the invention characterized in that the foreign nucleic acid molecules stably integrated into the genome of the plant cell or the plant encoding for a hyaluronan synthase and/or coding for a protein having the enzymatic activity of a UDP-Glc-DH are linked to regulatory elements initiating the transcription in plant cells (promoters). These may be homologous or heterologous promoters. The promoters can be constitutive, tissue-specific, development-specific or regulated by external factors (for example after application of chemical substances, by action of abiotic factors, such as heat and/or cold, draught, disease, etc.). Here, nucleic acid molecules coding for a hyaluronan synthase or a protein having the enzymatic activity of a UDP-Glc-DH, which nucleic acid molecules are integrated into the genome of a genetically modified plant cell according to the invention or a genetically modified plant according to the invention, may in each case be linked to the same promoter, or the individual sequences may be linked to different promoters.

A preferred embodiment of the present invention relates to genetically modified plant cells according to the invention or genetically modified plants according to the invention where at least one foreign nucleic acid molecule, particularly preferably at least two foreign nucleic acid molecules, especially preferably three foreign nucleic acid molecules selected from the group consisting of nucleic acid molecules coding for a hyaluronan synthase or a protein having the enzymatic activity of a UDP-Glc-DH is (are) linked to a tissue-specific promoter. Preferred tissue-specific promoters are promoters which initiate transcription specifically in plant tuber, leaf, fruit or seed cells.

To express nucleic acid molecules coding for a hyaluronan synthase or a protein having the enzymatic activity of a UDP-Glc-DH, these are preferably linked to regulatory DNA sequences ensuring the transcription and plant cells. These include in particular promoters. In general, any promoter active in plant cells is suitable for the expression. Here, the promoter may be chosen such that expression is constitutively or only in a certain tissue, at a certain point of the development of the plant or at a point in time determined by external factors. Both in respect to the plant and in respect of the nucleic acid molecule to be expressed, the promoter may be homologous or heterologous.

Suitable promoters are, for example, the promoter of 35S RNS of the cauliflower mosaic virus or the ubiquitin promoter from corn or the Cestrum YLCV (Yellow Leaf Curling Virus; WO 01 73087; Stavolone et al., 2003, Plant Mol. Biol. 53, 703-713) for a constitutive expression, the patatingen promoter B33 (Rocha-Sosa et al., EMBO J. 8 (1989), 23-29) for a tuber-specific expression in potatoes or a fruit-specific promoter for tomato, such as, for example, the polygalacturonase promoter from tomato (Montgomery et al., 1993, Plant Cell 5, 1049-1062) or the E8 promoter from tomato (Metha et al., 2002, Nature Biotechnol. 20(6), 613-618) or the ACC oxidase promoter from peach (Moon and Callahan, 2004, J. Experimental Botany 55 (402), 1519-1528) or a promoter which ensures expression only in photosynthetically active tissues, for example the ST-LS1 promoter (Stockhaus et al., Proc. Natl. Acad. Sci. USA 84 (1987), 7943-7947; Stockhaus et al., EMBO J. 8 (1989), 2445-2451) or for an endosperm-specific expression the HMWG promoter from wheat, the USP promoter, the phaseolin promoter, promoters of zein genes from corn (Pedersen et al., Cell 29 (1982), 1015-1026; Quatroccio et al., Plant Mol. Biol. 15 (1990), 81-93), the glutelin promoter (Leisy et al., Plant Mol. Biol. 14 (1990), 41-50; Zheng et al., Plant J. 4 (1993), 357-366; Yoshihara et al., FEBS Lett. 383 (1996), 213-218), a shrunken-1 promoter (Werr et al., EMBO J. 4 (1985), 1373-1380), a globulin promoter (Nakase et al, 1996, Gene 170(2), 223-226) or a prolamin promoter (Qu and Takaiwa, 2004, Plant Biotechnology Journal 2(2), 113-125). However, it is also possible to use promoters which are only active at a point in time determined by external factors (see, for example, WO 9307279). Of particular interest here may be promoters of heat-shock proteins which permit a simple induction. It is furthermore possible to use seed-specific promoters, such as, for example, the USP promoter from Vicia faba which ensures a seed-specific expression in Vicia faba and other plants (Fiedler et al., Plant Mol. Biol. 22 (1993), 669-679; Baumlein et al., Mol. Gen. Genet. 225 (1991), 459-467).

The use of promoters present in the genome of algae-infecting viruses are also suitable for expressing nucleic acid sequences in plants (Mitra et al., 1994, Biochem. Biophys Res Commun 204(1), 187-194; Mitra and Higgins, 1994, Plant Mol Biol 26(1), 85-93, Van Etten et al., 2002, Arch Virol 147,1479-1516).

In the context of the present invention, the term “tissue specific” is to be understood as meaning the substantial limitation of a manifestation (for example initiation of transcription) to a certain tissue.

In the context of the present invention, the terms “tuber, fruit or seed cell” are to be understood as meaning all cells present in a tuber, a fruit or in a seed.

In the context of the present invention, the term “homologous promoter” is to be understood as meaning a promoter which is naturally present in plant cells or plants used for the preparation of genetically modified plant cells according to the invention or genetically modified plants according to the invention (homologous with respect to the plant cell or the plant) or as meaning a promoter which regulates the regulation of the expression of a gene in the organism from which the sequence was isolated (homologous with respect to the nucleic acid molecule to be expressed).

In the context of the present invention, the term “heterologous promoter” is to be understood as meaning a promoter which is not naturally present in plant cells or plants used for the preparation of genetically modified plant cells according to the invention or genetically modified plants according to the invention (heterologous with respect to the plant cell or plant) or as meaning a promoter which is, in the organism from which a nucleic acid sequence to be expressed was isolated, not naturally present for regulating the expression of said nucleic acid sequence (heterologous with respect to the nucleic acid molecule to be expressed).

Also present may be a termination sequence (polyadenylation signal) which serves to add a poly-A tail to the transcript. The poly-A tail is thought to act in stabilizing the transcripts. Such elements are described in the literature (cf. Gielen et al., EMBO J. 8 (1989), 23-29) and can be exchanged as desired.

It is also possible for intron sequences to be present between the promoter and the coding region. Such intron sequences may lead to stability of expression and in increased expression in plants (Callis et al., 1987, Genes Devel. 1, 1183-1200; Luehrsen, and Walbot, 1991, Mol. Gen. Genet. 225, 81-93; Rethmeier et al., 1997; Plant Journal 12(4), 895-899; Rose and Beliakoff, 2000, Plant Physiol. 122 (2), 535-542; Vasil et al., 1989, Plant Physiol. 91, 1575-1579; XU et al., 2003, Science in China Series C Vol. 46 No. 6, 561-569). Suitable intron sequences are, for example, the first intron of the shl gene from corn, the first intron of the poly-ubiquitin gene 1 from corn, the first intron of the EPSPS gene from rice or one of the first two introns of the PAT1 gene from Arabidopsis.

The present invention also relates to plants comprising genetically modified plant cells according to the invention. Such plants may be produced by regeneration from genetically modified plant cells according to the invention.

The present invention also relates to processible or consumable parts of genetically modified plants according to the invention comprising genetically modified plant cells according to the invention.

In the context of the present invention, the term “processible parts” is to be understood as meaning plant parts which are used for preparing foodstuff or feedstuff, which are used as a raw material source for industrial processes, as a raw material source for the preparation of pharmaceutical products or as a raw material source for the preparation of cosmetic products.

In the context of the present invention, the term “consumable parts” is to be understood as meaning plant parts which serve as food for man or are used as animal feed.

The present invention also relates to a propagation material of genetically modified plants according to the invention comprising genetically modified plant cells according to the invention.

Here, the term “propagation material” comprises those components of the plant which are suitable for generating progeny via the vegetative or generative route. Suitable for vegetative propagation are, for example, cuttings, callus cultures, rhizomes or tubers. Other propagation material includes, for example, fruits, seeds, seedling, protoplasts, cell cultures, etc. The propagation material preferably takes the form of tubers, fruits or seeds.

In a further embodiment, the present invention relates to harvestable plant parts of genetically modified plants according to the invention, such as fruits, storage and other roots, flowers, buds, shoots, leaves or stalks, preferably seeds, fruits or tubers, these harvestable parts comprising genetically modified plant cells according to the invention.

Preferably, the present invention relates to propagation material according to the invention or harvestable parts of plants according to the invention comprising hyaluronan. Particularly preferred is propagation material according to the invention or harvestable parts of plants according to the invention which synthesize hyaluronan.

In the context of the present invention, the term “potato plant” or potato” is to be understood as meaning plant species of the genus Solanum, particularly tuber-producing species of the genus Solanum and in particular Solanum tuberosum.

In the context of the present invention, the term “tomato plant” or “tomato” is to be understood as meaning plant species of the genus Lycopersicon, in particular Lycopersicon esculentum.

The further advantage of the present invention is that harvestable parts, propagation material, processible parts or consumable parts of genetically modified plants according to the invention comprise more hyaluronan than hyaluronan-synthesizing transgenic plants described in the literature. Accordingly, genetically modified plants according to the invention are not only particularly suitable for use as raw material from which hyaluronan may be isolated but can also be used directly as foodstuff/feedstuff or for preparing foodstuff/feedstuff having a prophylactic or therapeutic character (for example for osteoarthritis prophylaxis, U.S. Pat. No. 6,607,745). Since genetically modified plants according to the invention have a higher hyaluronan content than the plants described in the literature, the preparation of such foodstuff/feedstuff requires lower amounts of harvestable parts, propagation material, processible parts or consumable parts of genetically modified plants according to the invention. If consumable parts of genetically modified plants according to the invention are consumed, for example, directly as a so-called “nutraceutical”, it is possible to achieve a positive effect even by ingesting relatively small amounts of substance. This may be of particular significance inter alia in the production of animal feed, since animal feed having too high a content of plant components is unsuitable as feedstuff for various animal species.

By virtue of the high capacity of hyaluronan to bind water, harvestable parts, propagation material, processible parts or consumable parts of genetically modified plants according to the invention furthermore have the advantage that less thickeners are required when solidified foodstuff/feedstuff is produced. Thus, for example, the production of jelly requires less sugar, which is associated with an additional positive effect on health. In the production of foodstuff/feedstuff requiring the dehydration of the crude plant material, the advantage of using harvestable parts, propagation material, processible parts or consumable parts of genetically modified plants according to the invention consists in the fact that less water has to be removed from the plant material in question, resulting in lower production costs and, owing to more gentle preparation methods (for example lower and/or shorter input of heat), an elevated nutritional value of the foodstuff/feedstuff in question. Thus, for example, in the production of tomato ketchup less energy has to be introduced in order to achieve the desired consistency.

The present invention furthermore provides a process for preparing a plant which synthesizes hyaluronan, which comprises
a) genetically modifying a plant cell, where the genetic modification comprises steps i to ii below
   i) introduction of a foreign nucleic acid molecule encoding for a hyaluronan synthase into the plant cell
   ii) introduction of a genetic modification into the plant cell, the genetic modification resulting in an increase of the activity of a protein having the enzymatic activity of a UDP-Glc-DH compared to corresponding not genetically modified wild-type plant cells
      where steps i to ii can be carried out in any order, individually, or any combinations of steps i to ii can be carried out simultaneously

b) regenerating a plant from plant cells from step a);

c) generating, if appropriate, further plants using the plants according to step b), where, if appropriate, plant cells are isolated from plants according to step b) i or b) ii and the process steps a) to c) are repeated until a plant is generated which has a foreign nucleic acid molecule coding for a hyaluronan synthase and has an increased activity of a protein having the enzymatic activity of a UDP-Glc-DH compared to corresponding not genetically modified wild-type plant cells.

The present invention preferably relates to processes for preparing a plant which synthesizes hyaluronan which comprises
a) genetically modifying a plant cell, where the genetic modification comprises steps i to ii below in any order, or any combinations of steps i to ii below may be carried out individually or simultaneously,
   i) introduction of a foreign nucleic acid molecule encoding for a hyaluronan synthase into the plant cell
   ii) introduction of a genetic modification into the plant cell, the genetic modification resulting in an increase of the activity of a protein having the enzymatic activity of a UDP-Glc-DH compared to corresponding not genetically modified wild-type plant cells
b) regenerating a plant from plant cells comprising the genetic modification according to steps
   i) a)i
   ii) a) ii
   iii) a) i and a) ii,
c) introducing into plant cells of plants according to step
   i) b) i a genetic modification according to step a) ii,
   ii) b) ii a genetic modification according to step a) i, and regenerating a plant
d) generating, if appropriate, further plants with the aid of the plants obtained according to any of steps b) iii or c) i or c) ii.

The genetic modifications introduced according to step a) into the plant cell may in principle be any type of modification resulting in an increased activity of a protein having the enzymatic activity of a UDP-Glc-DH.

The regeneration of the plants according to step b) and, if appropriate, step c) of the processes according to the invention can be carried out using methods known to the person skilled in the art (described, for example, in “Plant Cell Culture Protocols”, 1999, edited by R. D. Hall, Humana Press, ISBN 0-89603-549-2). The generation of further plants (depending on the process according to step c) or step d)) of the processes according to the invention can be carried out, for example, by vegetative propagation (for example via cuttings, tubers or via callus culture and regeneration of intact plants) or via generative propagation. In this context, generative propagation preferably takes place under controlled conditions, i.e. selected plants with specific characteristics are hybridized with one another and multiplied. The selection preferably takes place in such a manner that the further plants (depending on the process generated according to step c) or step d)) comprise the modifications introduced in the preceding steps.

In processes according to the invention for preparing plants which synthesize hyaluronan, the genetic modifications for generating the genetically modified plant cells according to the invention can be carried out simultaneously or in successive steps. Here, it is immaterial whether the same method as for the genetic modification introducing a foreign nucleic acid molecule coding for a hyaluronan synthase into the plant cell is used for successive genetic modifications resulting in an increased activity of a protein having the enzymatic activity of a UDP-Glc-DH.

In a further embodiment of processes according to the invention for preparing a plant which synthesizes hyaluronan, the genetic modification consists in the introduction of a foreign nucleic acid molecule into the genome of the plant cell, where the presence or the expression of the foreign nucleic acid molecule(s) results in an increased activity of a protein having the enzymatic activity of a UDP-Glc-DH in the plant cell.

As already described above for the foreign nucleic acid molecules introduced for genetic modification into the plant cell or plant, what is introduced in step a) of the processes according to the invention for preparing a plant which synthesizes hyaluronan may be an individual nucleic acid molecule or a plurality of nucleic acid molecules. Thus, the foreign nucleic acid molecules coding for a hyaluronan synthase and/or coding for a protein having the enzymatic activity of a UDP-Glc-DH may be present together on a single nucleic acid molecule, or they may be present on separate nucleic acid molecules. If the nucleic acid molecules coding for a hyaluronan synthase and coding for a protein having the activity of are present on a plurality of nucleic acid molecules, these nucleic acid molecules may be introduced simultaneously or in successive steps into a plant cell.

Furthermore, to introduce a foreign nucleic acid molecule in the practice of processes according to the invention for preparing a plant which synthesizes hyaluronan, it is possible to use, instead of a wild-type plant cell or wild-type plant, mutant cells or mutants which are distinguished in that they already have an increased activity of a protein having the enzymatic activity of a UDP-Glc-DH. If the mutant cell or the mutant already has an increased activity of a protein having the enzymatic activity of a UDP-Glc-DH compared to the corresponding wild-type plant cells or wild-type plants, it is sufficient for carrying out a process according to the invention for producing a plant which synthesizes hyaluronan to introduce into said mutant cell or mutant a foreign nucleic acid molecule coding for a hyaluronan synthase.

All said further above concerning the use of mutants for the preparation of genetically modified plant cells according to the invention or genetically modified plants according to the invention applies here in an analogous manner.

In preferred embodiments, the present invention relates to processes according to the invention for producing a plant which synthesizes hyaluronan, wherein the nucleic acid molecule coding for a hyaluronan synthase in step a) is selected from the group consisting of:
a) nucleic acid molecules characterized in that they code for a viral hyaluronan synthase,
b) nucleic acid molecules characterized in that they code for a hyaluronan synthase of a Chlorella-infecting virus,
c) nucleic acid molecules characterized in that they code for a hyaluronan synthase of a Paramecium bursaria Chlorella Virus 1,
d) nucleic acid molecules characterized in that they code for a hyaluronan synthase of a Paramecium bursaria Chlorella 1 Virus 1 of strain H1,
e) nucleic acid molecules characterized in that the codons of the nucleic acid molecule coding for a hyaluronan synthase are modified compared to the codons of the nucleic acid molecule which codes for the hyaluronan synthase in the parent organism of the hyaluronan synthase,
f) nucleic acid molecules characterized in that the codons of the hyaluronan synthase have been modified thus that they are adapted to the frequency of the use of the codons of the plant cell or of the plant into whose genome they are to be integrated or are integrated,
g) nucleic acid molecules characterized in that they code for a hyaluronan synthase having the amino acid sequence shown under SEQ ID NO 2 or that they code for a hyaluronan synthase whose amino acid sequence is at least 70%, preferably at least 80%, particularly preferably at least 90% and especially preferably at least 95% identical to the amino acid sequence shown under SEQ ID NO 2,
h) nucleic acid molecules characterized in that they code for a protein whose amino acid sequence can be derived from the coding region of the nucleic acid sequence inserted into plasmid DSM16664 or that it codes for a protein whose amino acid sequence is at least 70%, preferably at least 80%, particularly preferably at least 90% and especially preferably at least 95% identical to the amino acid sequence which can be derived from the coding region of the nucleic acid sequence inserted into plasmid DSM16664,
i) nucleic acid molecules comprising a nucleic acid sequence shown under SEQ ID NO 1 or SEQ ID NO 3 or being at least 70%, preferably at least 80%, with preference at least 90% and especially preferably at least 95% identical to the nucleic acid sequence shown under SEQ ID NO 1 or SEQ ID NO 3,
j) nucleic acid molecules comprising the nucleic acid sequence inserted into plasmid DSM16664 or being at least 70%, preferably at least 80%, with preference at least 90% and especially preferably at least 95% identical to the nucleic acid sequence inserted into plasmid DSM16664,
k) nucleic acid molecules coding for a hyaluronan synthase, where the nucleic acid sequences coding for the hyaluronan synthase are linked to regulatory elements (promoter) which initiate the transcription in plant cells or
l) nucleic acid molecules according to k) where the promoters are tissue-specific promoters, particularly preferably promoters which initiate the initiation of transcription specifically in plant tuber, fruit or seed cells.

In preferred embodiments, the present invention relates to processes according to the invention for producing a plant which synthesizes hyaluronan, where the foreign nucleic acid molecule coding for a protein having the activity of a UDP-Glc-DH is selected from the group consisting of:
a) nucleic acid molecules characterized in that they code for a protein having the activity of a UDP-Glc-DH originating from viruses, bacteria, animals or plants,
b) nucleic acid molecules characterized in that they code for a protein having the activity of a UDP-Glc-DH of a Chlorella-infecting virus,
c) nucleic acid molecules characterized in that they code for a protein having the activity of a UDP-Glc-DH of a Paramecium bursaria Chlorella virus,
d) nucleic acid molecules characterized in that the codons of the nucleic acid molecule coding for a protein having the activity of a UDP-Glc-DH are modified compared to the codons of a nucleic acid molecule coding for the corresponding protein having the activity of a UDP-Glc-DH of the parent organism,
e) nucleic acid molecules characterized in that the codons of the protein havingf the activity of a UDP-Glc-DH are modified thus that they are adapted to the frequency of the use of the codons of the plant cell or of the plant into whose genome they are to be integrated or are integrated,
f) nucleic acid molecules coding for a protein having the amino acid sequence shown under SEQ ID NO 5;
g) nucleic acid molecules coding for a protein whose sequence is at least 70%, preferably at least 80%, with preference at least 90% and especially preferably at least 95% identical to the amino acid sequence shown under SEQ ID NO 5;
h) nucleic acid molecules comprising the nucleotide sequence shown under SEQ ID NO 4 or under a sequence complementary thereto or the nucleotide sequence shown under SEQ ID NO 6 or a sequence complementary thereto;
i) nucleic acid molecules which are at least at least 70%, preferably at least 80%, with preference at least 90% and especially preferably at least 95% identical to the nucleic acid sequences described under h);
j) nucleic acid molecules which hybridize under stringent conditions with at least one strand of the nucleic acid molecules described under f) or h);
k) nucleic acid molecules whose nucleotide sequence differs from the sequence of the nucleic acid molecules mentioned under f) or h) owing to the degeneration of the genetic code; and
l) nucleic acid molecules which are fragments, allelic variants and/or derivatives of the nucleic acid molecules mentioned under a), b), c), d), e), f) or h),
m) nucleic acid molecules coding for a protein having the activity of a UDP-Glc-DH, where the nucleic acid sequences coding for a protein having the activity of a UDP-Glc-DH are linked to regulatory elements (promoter) which initiate the transcription in plant cells or
n) nucleic acid molecules according to m), where the promoters are tissue-specific promoters, particularly preferably promoters which initiate the initiation of transcription specifically in plant tuber, leaf, fruit or seed cells.

In a further preferred embodiment, processes according to the invention for producing a plant which synthesizes hyaluronan are used for producing genetically modified plants according to the invention.

The present invention also provides plants obtainable by a process according to the invention for producing a plant which synthesizes hyaluronan.

The present invention furthermore relates to a process for producing hyaluronan which comprises the step of extracting hyaluronan from genetically modified plant cells according to the invention, from genetically modified plants according to the invention, from propagation material according to the invention, from harvestable plant parts according to the invention or from plants or parts of these plants obtainable by a process according to the invention for producing plants which synthesize hyaluronan.

Preferably, such a process also comprises the step of harvesting the cultivated genetically modified plant cells according to the invention, the genetically modified plants according to the invention, the propagation material according to the invention, the harvestable plant parts according to the invention, the processible plant parts according to the invention prior to extracting the hyaluronan, and particularly preferably furthermore the step of cultivating genetically modified plant cells according to the invention or genetically modified plants according to the invention prior to harvesting.

In contrast to bacterial or animal tissues, plant tissues have no hyaluronidases and do not contain any hyaladherins. Accordingly, as already described above, extraction of hyaluronan from plant tissues is possible using relatively simple methods. If required, the aqueous extracts, described above, of plant cells or tissues containing hyaluronan can be purified further using methods known to the person skilled in the art, such as, for example, repeated precipitation with ethanol. A preferred method for purifying hyaluronan is described under General Methods item 3.

The processes already described for extracting hyaluronan from genetically modified plant cells according to the invention or genetically modified plants according to the invention are also suitable for isolating hyaluronan from propagation material according to the invention, from harvestable plant parts according to the invention or from plants or parts of these plants obtainable by a process according to the invention for preparing plants which synthesize hyaluronan.

The present invention also provides the use of genetically modified plant cells according to the invention, genetically modified plants according to the invention, propagation material according to the invention, harvestable plant parts according to the invention, processible plant parts according to the invention or plants obtainable by a process according to the invention for preparing hyaluronan.

The present invention furthermore relates to compositions comprising genetically modified plant cells according to the invention. Here, it is immaterial whether the plant cells are intact or no longer intact because they have been destroyed, for example, by processing. The compositions are preferably foodstuff or feedstuff, pharmaceutical or cosmetic products.

The present invention preferably provides compositions comprising components of genetically modified plant cells according to the invention, of genetically modified plants according to the invention, of propagation material according to the invention, of harvestable plant parts according to the invention or of plants obtainable by a process according to the invention and comprising recombinant nucleic acid molecules, where the recombinant nucleic acid molecules are characterized in that they comprise nucleic acid molecules coding for a hyaluronan synthase and proteins having the enzymatic activity of a UDP-Glc-DH.

A stable integration of foreign nucleic acid molecules into the genome of a plant cell or plant results in the foreign nucleic acid molecules being flanked after integration into the genome of a plant cell or plant by genomic plant nucleic acid sequences.

Accordingly, in a preferred embodiment, compositions according to the invention are characterized in that the recombinant nucleic acid molecules present in the composition according to the invention are flanked by genomic plant nucleic acid sequences.

Here, the genomic plant nucleic acid sequences may be any sequences naturally present in the genome of the plant cell or plant used for preparing the composition.

The recombinant nucleic acid molecules present in the compositions according to the invention may be individual or various recombinant nucleic acid molecules which nucleic acid molecules coding for a hyaluronan synthase and proteins having the enzymatic activity of a UDP-Glc-DH are present on a single nucleic acid molecule, or those where the nucleic acid molecules may be present on separate recombinant nucleic acid molecules. Depending on how the nucleic acid molecules coding for a hyaluronan synthase or coding for a protein having the enzymatic activity of a UDP-Glc-DH are present in a composition according to the invention, they may be flanked by identical or different genomic plant nucleic acid sequences.

That compositions according to the invention comprise recombinant nucleic acid molecules may be demonstrated using methods known to the person skilled in the art, such as, for example, methods based on hybridization or, preferably, using methods based on PCR (polymerase chain reaction).

Preferably, compositions according to the invention comprise at least 0.005%, with preference at least 0.01%, particularly preferably at least 0.05% and especially preferably at least 0.1% of hyaluronan.

As already mentioned above, it is possible to use genetically modified plant cells according to the invention, genetically modified plants according to the invention, propagation material according to the invention, harvestable plant parts according to the invention, processible plant parts according to the invention, consumable plant parts according to the invention or plants obtainable by a process according to the invention to prepare foodstuff or feedstuff. However, use as raw materials for industrial applications is also possible, without hyaluronan having to be isolated. Thus, for example, genetically modified plants according to the invention or parts of genetically modified plants according to the invention can be applied to areas under agricultural cultivation to achieve increased water binding of the soil. Furthermore, genetically modified plants according to the invention or genetically modified plant cells according to the invention can be used for preparing drying agents (for example for use when shipping moisture-sensitive items) or as absorbers of liquids (for example in nappies or for absorbing spilt aqueous liquids). For such applications, it is possible to use entire genetically modified plants according to the invention, parts of genetically modified plants according to the invention or comminuted (for example ground) genetically modified plants according to the invention or plant parts according to the invention, as required. Suitable for applications in which ground plants or plant parts are used are in particular plant parts containing hyaluronan, but only a low proportion of water. These are preferably grains of cereal plants (corn, rice, wheat, rye, oats, barley, sago or sorghum). Since genetically modified plant cells according to the invention and genetically modified plants according to the invention have a higher hyaluronan content than transgenic plants described in the literature, compared to these less material has to be used for industrial applications when use is made of genetically modified plant cells according to the invention or genetically modified plants according to the invention.

The present invention also provides processes for preparing a composition according to the invention, where genetically modified plant cells according to the invention, genetically modified plants according to the invention, propagation material according to the invention, harvestable plant parts according to the invention, processible plant parts according to the invention, consumable plant parts according to the invention or plants obtainable by a process according to the invention for producing a plant which synthesizes hyaluronan are used. The processes for preparing a composition according to the invention are preferably processes for preparing foodstuff or feedstuff, processes for preparing a pharmaceutical product or processes for preparing a cosmetic product.

Process for preparing foodstuff or feedstuff are known to the person skilled in the art. Processes for using genetically modified plants according to the invention or plant parts according to the invention in industrial areas are also known to the person skilled in the art and include inter alia comminuting or grinding of genetically modified plants according to the invention or plant parts according to the invention; however, they are not exclusively limited thereto. Some of the advantages resulting from using subject-matters according to the invention for preparing foodstuff/feedstuff or for use in industrial areas have already been described above.

A process according to the invention for preparing a composition is particularly preferably a process for preparing a composition which comprises hyaluronan.

Compositions obtainable by a process for preparing a composition according to the invention are likewise provided by the present invention.

The present invention also relates to the use of genetically modified plant cells according to the invention, genetically modified plants according to the invention, propagation material according to the invention, harvestable plant parts according to the invention, processible plant parts according to the invention, consumable plant parts according to the invention or plants obtainable by a process according to the invention for producing a plant which synthesizes hyaluronan for preparing a composition according to the invention. Preference is given to the use of genetically modified plant cells according to the invention, genetically modified plants according to the invention, propagation material according to the invention, harvestable plant parts according to the invention, processible plant parts according to the invention, consumable plant parts according to the invention or of plants obtainable by a process according to the invention for producing a plant which synthesizes hyaluronan for preparing foodstuff or feedstuff, for preparing a pharmaceutic or for preparing a cosmetic product.

Description of the Sequences
SEQ ID NO 1: Nucleic acid sequence coding for a hyaluronan synthase of Paramecium bursaria Chlorella Virus 1.
SEQ ID NO 2: Amino acid sequence of a hyaluronan synthase of the Paramecium bursaria Chlorella Virus 1. The amino acid sequence shown can be derived from SEQ ID NO 1.
SEQ ID NO 3: Synthetic nucleic acid sequence coding for a hyaluronan synthase of Paramecium bursaria Chlorella Virus 1. The synthesis of the codons of the sequence shown was carried out such that it is adapted to the use of codons in plant cells. The nucleic acid sequence shown codes for a protein having the amino acid sequence shown under SEQ ID NO 2.
SEQ ID NO 4: Nucleic acid sequence coding for a protein having the activity of a UDP-Glc-DH of Paramecium bursaria Chlorella Virus 1.
SEQ ID NO 5: Amino acid sequence of a protein having the activity of a UDP-Glc-DH of Paramecium bursaria Chlorella Virus 1. The amino acid sequence shown can be derived from SEQ ID NO 4.
SEQ ID NO 6: Synthetic nucleic acid sequence coding for a protein having the activity of a UDP-Glc-DH of Paramecium bursaria Chlorella Virus 1. The synthesis of the codons of the sequence shown was carried out such that it was adapted to the use of codons in plant cells. The nucleic acid sequence shown codes for a protein having the amino acid sequence shown under SEQ ID NO 5.
SEQ ID NO 7: Synthetic oligonucleotide, used in example 1.
SEQ ID NO 8: Synthetic oligonucleotide, used in example 1.

### DESCRIPTION OF THE FIGURES

FIG. 1: Shows a calibration curve and the corresponding equation of the regression line used for calculating the hyaluronan content in plant tissue. The calibration curve was established with the aid of the commercial test kit (Hyaluronic Acid (HA) test kit from Corgenix Inc., Colorado,. USA, Prod. No. 029-001) and the standard solutions supplied therewith.

### GENERAL METHODS

Methods which can be used in connection with the present invention are described below. These methods are specific embodiments; however, the present invention is not limited to these methods. It is known to the person skilled in the art that the invention can be carried out in the same manner by modifying the methods described and/or by replacing individual methods or parts of methods by alternative methods or alternative parts of methods.

1. Transformation of Potato Plants

Potato plants were transformed with the aid of Agrobacterium, as described in Rocha-Sosa et al. (EMBO J. 8, (1989), 23-29).

2. Isolation of Hyaluronan from Plant Tissue

To detect the presence of hyaluronan and to determine the hyaluronan content in plant tissue, plant material was worked up as follows: 200 μl of water (demineralized, conductivity≧18 MΩ) were added to about 0.3 g of tuber material, and the mixture was comminuted in a laboratory oscillating ball mill (MM200, from Retsch, Germany) (30 sec at 30 Hz) A further 800 μl of water (demineralized, conductivity≧18 MΩ) was then added, and the mixture was mixed well (using, for example, a Vortex mixer). Cell debris and insoluble components were separated from the supernatant by centrifuging at 16 000×g for 5 minutes.

3. Purification of Hyaluronan

About 100 grams of tubers were peeled, cut into pieces of a size of about 1 cm³ and, after addition of 100 ml of water (demineralized, conductivity≧18 MΩ) comminuted in a Warring blender at maximum speed for about 30 seconds. The cell debris was then removed using a tea sieve. The cell debris that had been removed was resuspended in 300 ml of water (demineralized, conductivity≧18 MΩ) and again removed using a tea sieve. The two suspensions obtained (100 ml+300 ml) were combined and centrifuged at 13 000×g for 15 minutes. NaCl was added to the centrifugation supernatant obtained until a final concentration of 1% had been reached. After the NaCl had gone into solution, precipitation was carried out by addition of twice the volume of ethanol followed by thorough mixing and incubation at −20° C. overnight. The mixture was then centrifuged at 13 000×g for 15 minutes. The sedimented precipitate obtained after this centrifugation was dissolved in 100 ml of buffer (50 mM TrisHCl, pH 8, 1 mM CaCl₂) and proteinase K was then added to a final concentration of 100 μg/ml and the solution was incubated at 42° C. for 2 hours. This was followed by 10 minutes of incubation at 95° C. Once more, NaCl was added to this solution until a final concentration of 1% had been reached. After the NaCl had gone into solution, another precipitation was carried out by addition of twice the volume of ethanol, thorough mixing and incubation at −20° C. for about 96 hours. This was followed by 15 minutes of centrifugation at 13 000×g. The sedimented precipitate obtained after this centrifugation was dissolved in 30 ml of water (demineralized, conductivity≧18 MΩ), and once more, NaCl was added to a final concentration of 1%. By adding twice the volume of ethanol, thorough mixing and incubation at −20° C. overnight, another precipitation was carried out. The precipitate obtained after subsequent centrifugation at 13 000×g for 15 minutes was dissolved in 20 ml of water (demineralized, conductivity≧18 MΩ).

Further purification was carried out by centrifugal filtration. To this end, in each case 5 ml of the dissolved precipitate were applied to a membrane filter (CentriconAmicon, pore width 10 000 NMWL, Prod. No. UCF8 010 96), and the sample was centrifuged at 2200×g until only about 3 ml of the solution above the filter remained. Two more times, in each case 3 ml of water (demineralized, conductivity≧18 MΩ) were then added to the solution above the membrane and in each case re-centrifuged under identical conditions until, at the end, only about 3 ml of the solution above the filter remained. The solutions still present above the membrane after centrifugal filtration were taken off, and the membrane was rinsed repeatedly (three to five times) with about 1.5 ml of water (demineralized, conductivity≧18 MΩ). All solutions which were still present above the membrane and the solutions obtained from rinsing were combined, NaCl was added to a final concentration of 1%, after the NaCl had gone into solution, twice the volume of ethanol was added, the sample was mixed and a precipitate was obtained by storage at −20° C. ovemight. The precipitate obtained after subsequent centrifugation at 13 000×g for 15 minutes was dissolved in 4 ml of water (demineralized, conductivity≧18 MΩ) and then freeze-dried (24 hours under a pressure of 0.37 mbar, freeze drying apparatus Christ Alpha 1-4 from Christ, Osterode, Germany).

4. Detection of Hyaluronan and Determination of the Hyaluronan Content

Hyaluronan was detected using a commercial test (hyaluronic acid (HA) test kit from Corgenix, Inc., Colorado, USA, Prod. No. 029-001) according to the instructions of the manufacturer which are herewith incorporated into the description by way of reference. The test principle is based on the availability of a protein which binds specifically to hyaluronan (HABP) and is carried out similarly to an ELISA, where a color reaction indicates the hyaluronan content in the sample examined. Accordingly, for the quantitative determination of hyaluronan, the samples to be measured should be employed in a concentration such that it is within the stated limits (for example: dilution of the sample in question or use of less water for extracting hyaluronan from plant tissue, depending on whether a limit was exceeded or not reached).

In parallel batches, aliquots of the samples to be determined were initially subjected to hyaluronidase digestion and then measured using the commercial test (hyaluronic acid (HA) test kit from Corgenix, Inc., Colorado, USA, Prod. No. 029-001). Hyaluronidase digestion was carried out using 400 μl of potato tuber extract in hyaluronidase buffer (0.1 M potassium phosphate buffer, pH 5.3; 150 mM NaCl) by adding 5 μg (˜3 units) of hyaluronidase (hyaluronidase type III from Sigma, Prod. No. H 2251) and incubating at 37° C. for 30 min.

In each case in a dilution of 1:10, all samples were then used for determining the hyaluronan content.

5. Determination of the Activity of a UDP-Glc-DH

The activity of a protein having the activity of UDP-Glc-DH is determined as described in Spicerl et al. (1998, J. Bacteriol. 273 (39), 25117-25124).

### EXAMPLES

1. Preparation of the Plant Expression Vector IR 47-71

The plasmid pBinAR is a derivative of the binary vector plasmid pBin19 (Bevan, 1984, Nuci Acids Res 12: 8711-8721) which was constructed as follows:

A fragment of a length of 529 bp which comprised the nucleotides 6909-7437 of the 35S promoter of the cauliflower mosaic virus was isolated as EcoR I/Kpn I fragment from the plasmid pDH51 (Pietrzak et al, 1986 Nucleic Acids Res. 14, 5858) and ligated between the EcoR I and Kpn I restriction sites of the polylinker of pUC18. In this manner, the plasmid pUC18-35S was formed. Using the restriction endonucleases Hind III and Pvu II, a fragment of a length of 192 bp which included the polyadenylation signal (3′ terminus) of the Octopin Synthase gene (gene 3) of the T-DNA of the Ti plasmid pTiACH5 (Gielen et al, 1984, EMBO Journal 3, 835-846) (nucleotides 11 749-11 939) was isolated from the plasmid pAGV40 (Herrera-Estrella et al, 1983 Nature, 303, 209-213). Following addition of Sph I linkers to the Pvu II restriction site, the fragment was ligated between the Sph I and Hind III restriction sites of pUC18-35S. This gave the plasmid pA7. Here, the entire polylinker comprising the 35S promoter and Ocs terminator was removed using EcoR I and Hind III and ligated into the appropriately cleaved vector pBin19. This gave the plant expression vector pBinAR (Höfgen and Willmitzer, 1990, Plant Science 66, 221-230).

The promoter of the patatin gene B33 from Solanum tuberosum (Rocha-Sosa et al., 1989, EMBO J. 8, 23-29) was, as Dra I fragment (nucleotides −1512-+14), ligated into the Sst I-cleaved vector pUC19 whose ends had been blunted using T4-DNA polymerase. This gave the plasmid pUC19-B33. From this plasmid, the B33 promoter was removed using EcoR I and Sma I and ligated into the appropriately restricted vector pBinAR. This gave the plant expression vector pBinB33.

To facilitate further cloning steps, the MCS (Multiple Cloning Site) was extended. To this end, two complementary oligonucleotides were synthesized, heated at 95° C. for 5 minutes, slowly cooled to room temperature to allow good annealing and cloned into the Sal I and Kpn I restriction sites of pBinB33. The oligonucleotides used for this purpose had the following sequence: 5′-TCg ACA ggC CTg gAT CCT TAA TTA AAC TAg TCT CgA ggA gCT Cgg TAC-3′ 5′-CgA gCT CCT CgA gAC TAg TTT AAT TAA ggA TCC Agg CCT g-3′

The plasmid obtained was named IR 47-71.

2. Preparation of the Plant Expression Vector pBinARHyg

The fragment comprising the 35S promoter, the Ocs terminator and the entire Multiple Cloning Site was removed from pA7 using the restriction endonucleases EcoR I and Hind III and cloned into the vector pBIBHyg (Becker, 1990, Nucleic Acids Res. 18, 203) which had been cut using the same restriction endonucleases. The plasmid obtained was named pBinARHyg.

3. Synthesis of Nucleic Acid Molecules
a) Synthesis of nucleic acid molecules coding for a hyaluronan synthase of Paramecium bursaria Chlorella virus 1

The nucleic acid sequence coding for a hyaluronan synthase of Paramecium bursaria Chlorella virus 1 was synthesized by Medigenomix GmbH (Munich, Germany) and cloned into the vector pCR2.1 from Invitrogen (Prod. No. K2000-01). The plasmid obtained was named IC 323-215. The synthetic nucleic acid sequence coding for the HAS protein from Paramecium bursaria Chlorella Virus 1, is shown under SEQ ID NO 3. The corresponding nucleic acid sequence originally isolated from the Paramecium bursaria Chlorella virus 1 is shown under SEQ ID NO 1.
b) Synthesis of nucleic acid molecules coding for a protein having the activity of a UDP-Glc-DH of Paramecium bursaria Chlorella virus 1 The nucleic acid sequence coding for a protein having the activity of a UDP-Glc-DH of Paramecium bursaria Chlorella virus 1 was synthesized by Entelechon GmbH and cloned into the vector pCR4Topo from Invitrogen (Prod. No. K4510-20). The plasmid obtained was named IC 339-222. The synthetic nucleic acid sequence coding for the UDP-Glc-DH protein from Paramecium bursaria Chlorella virus 1, is shown under SEQ ID NO 6. The corresponding nucleic acid sequence originally isolated from Paramecium bursaria Chlorella virus 1 is shown under SEQ ID NO 4.
4. Preparation of the plant expression vector IC 341-222 which comprises a coding nucleic acid sequence for a hyaluronan synthase of Paramecium bursafia Chlorella virus 1

Using restriction digestion with BamH I and Xho I, nucleic acid molecules comprising the coding sequence of hyaluronan synthase were isolated from the plasmid IC 323-215 and cloned into the BamH I and Xho I restriction sites of the plasmid IR 47-71. The plant expression vector obtained was named IC 341-222.
5. Preparation of the plant expression vector IC 349-222 comprising coding nucleic acid sequences for a protein having the activity of a UDP-Glc-DH of Paramecium bursaria Chlorella virus 1

Using restriction digestion with BamH I and Kpn I, nucleic acid molecules comprising the coding sequence for a protein having the activity of a UDP-Glc-DH of Paramecium bursaria Chlorella virus 1 were isolated from the plasmid IC 339-222 and cloned into the plasmid pA7 which had been cut by means of the same restriction endonucleases. The plasmid obtained was named IC 342-222.

Nucleic acid molecules comprising the coding sequence for a protein having the activity of a UDP-Glc-DH of Paramecium bursaria Chlorella virus 1 were isolated from the plasmid IC 342-222 by restriction digestion with Xba I and Kpn I and cloned into the expression vector pBinARHyg which had been cut with Xba I and Kpn 1. The plasmid obtained was named IC 349-222.
6. Transformation of plants with plant expression vectors comprising nucleic acid molecules coding for a hyaluronan synthase

Potato plants (cv Désirée) were transformed using the plant expression vector IC 341-222, which comprises a coding nucleic acid sequence for a hyaluronan synthase from Paramecium bursaria Chlorella virus 1 under the control of the promoter of the patatin gene B33 from Solanum tuberosum (Rocha-Sosa et al., 1989, EMBO J. 8, 23-29) using the method given under General Methods item 1. The transgenic potato plants obtained, which were transformed with the plasmid IC 341-222, were named 365 ES.
7. Analysis of the transgenic plants transformed with plant expression vectors comprising nucleic acid molecules coding for a hyaluronan synthase

a) Construction of a Calibration Curve

A calibration curve was constructed using the standard solutions supplied with the commercial test kit (hyaluronic acid (HA) test kit from Corgenix, Inc., Colorado, USA, Prod. No. 029-001), according to the methods described by the manufacturer. To determine the extinction at 1600 ng/ml of hyaluronan, double the amount, based on the amount of supplied standard indicated by the manufacturer, comprising 800 ng/ml of hyaluronan was used. In each case, three independent measurement series were carried out, and the corresponding mean was determined. This gave the following calibration curve:

TABLE 1

| | Independent | | | | |
|---|---|---|---|---|---|
| Hyaluronan | individual measurements | | | | |
| concentration | E_{450 nm} | E_{450 nm} | E_{450 nm} | Mean | s.d. |
| 0 ng/ml | 0.100 | 0.096 | 0.096 | 0.097 | 0.002 |
| 50 ng/ml | 0.224 | 0.183 | 0.222 | 0.210 | 0.023 |
| 100 ng/ml | 0.396 | 0.263 | 0.377 | 0.345 | 0.072 |
| 200 ng/ml | 0.554 | 0.443 | 0.653 | 0.550 | 0.105 |
| 500 ng/ml | 1.231 | 0.850 | 1.221 | 1.101 | 0.217 |
| 800 ng/ml | 1.465 | 1.265 | 1.795 | 1.508 | 0.268 |
| 1600 ng/ml | 2.089 | 2.487 | 3.170 | 2.582 | 0.547 |

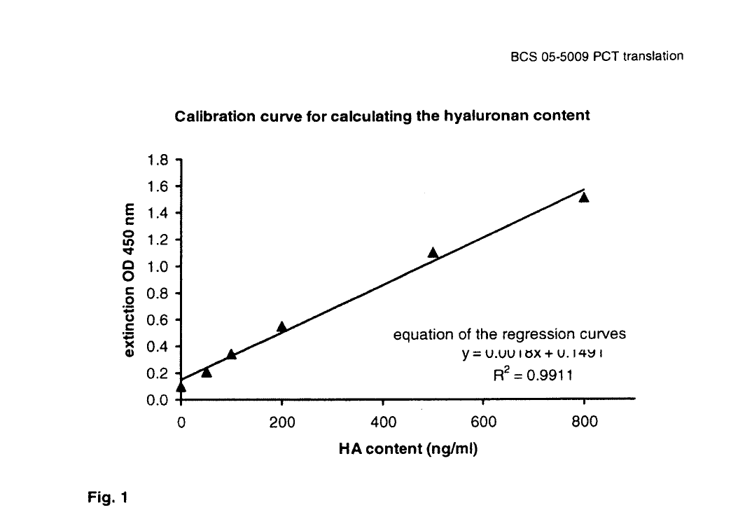

Values for constructing a calibration curve for the quantitative determination of the hyaluronan content in plant tissue. With the aid of software (Microsoft Office Excel 2002, SP2), the measured values obtained were entered into a diagram and the equation of the function of the trend line was determined (see FIG. 1). E_{450 nm} refers to the extinction at a wavelength of 450 nm, s.d. is the standard deviation of the calculated mean of the individual values.

b) Analysis of Potato Tubers of Lines 365 ES

In a greenhouse, individual plants of the line 365 ES were cultivated in soil in 6 cm pots.

In each case about 0.3 g of material of potato tubers of the individual plants was processed according to the method described under General Methods item 2. Using the method described under General Methods item 4, the amount of hyaluronan present in the respective plant extracts was determined, with the aid of the calibration curve shown in Example 7a) and FIG. 1. Here, the supernatant obtained after centrifugation was used in a dilution of 1:10 for determining the hyaluronan content. For selected plants, the following results were obtained:

TABLE 2

| | Weight of the | | Amount of | Hyaluronan based on |
|---|---|---|---|---|
| | plant material | Extinction | hyaluronan | the fresh weight of the |
| Name of the plant | employed [g] | E450 | [ng/ml] | plant material [μg/g] |
| 365 ES 13 | 0.297 | 2.746 | 14038 | 47 |
| 365 ES 74 | 0.306 | 4.000 | 20816 | 68 |
| Wild-type | 0.305 | 0.111 | n.d. | n.d. |

Amount of hyaluronan (in μg of hyaluronan per g of fresh weight) produced by independent transgenic plants of the line 365 ES. Column 1 refers to the plant from which tuber material was harvested (here, “wild-type” refers to untransformed plants which, however, have the genotype used as starting material for the transformation). Column 2 indicates the amount of tuber material of the plant in question used for determining the hyaluronan content. Column 3 contains the measured extinction of a 1:10 dilution of the respective plant extract. Column 4 was calculated with the aid of the regression line equation (see FIG. 1) taking into account the dilution factor, as follows: ((value column 3 − 0.149)/0.00185) × 10. Column 5 indicates the amount of hyaluronan based on the fresh weight used and was calculated as follows: (value column 4/value column 2)/1000. “n.d.” is amounts which are not detectable.

8. Transformation of hyaluronan-synthesizing plants with plant expression vectors comprising coding nucleic acid sequences for a protein having the activity of a UDP-Glc-DH

Potato plants of the lines 365 ES 13 and 365 ES 74 were in each case transformed with the plant expression vector 349-222 using the method given under General Methods item 1.

1. A genetically modified plant cell comprising a nucleic acid molecule coding for a hyaluronan synthase stably integrated into its genome and wherein said plant cell additionally has an increased activity of a protein having the activity of a UDP-glucose dehydrogenase (UDP-Glc-DH) compared to corresponding not genetically modified wild-type plant cells.

2. The genetically modified plant cell of claim 1, wherein the increased activity of a protein having the activity is caused by introduction of a foreign nucleic acid molecule into the plant cell.

3. The genetically modified plant cell of claim 1, wherein said foreign nucleic acid molecule codes for a protein having the enzymatic activity of a UDP-glucose dehydrogenase (UDP-Glc-DH).

4. The genetically modified plant cell of claim 1, wherein said cell synthesizes an increased amount of hyaluronan compared to plant cells having the activity of a hyaluronan synthase and no increased activity of a UDP-glucose dehydrogenase (UDP-Glc-DH).

5. A plant comprising genetically the modified plant cells claim 1.

6. Propagation material of the plants of claim 5.

7. Harvestable plant parts of the plants of claim 5.

8. A process for producing a plant which synthesizes hyaluronan comprising
a) genetically modifying a plant cell, wherein said genetic modification comprises
i) introduction of a foreign nucleic acid molecule encoding for a hyaluronan synthase into the plant cell;
ii) introduction of a genetic modification into the plant cell, wherein the genetic modification results in an increase of the activity of a protein having the enzymatic activity of a UDP-glucose dehydrogenase (UDP-Glc-DH) compared to corresponding non genetically modified wild-type plant cells wherein steps i to ii can be carried out in any order, individually, or any combinations of steps i to ii can be carried out simultaneously and
b) regenerating a plant from plant cells from step a);
c) optionally, generating, further plants using the plants according to step b), where plant cells are isolated from plants obtained according to steps b) i or b) ii and the process steps a) to c) are repeated until a plant is generated which has a foreign nucleic acid molecule coding for a hyaluronan synthase and has an increased activity of a protein having the enzymatic activity of a GFAT compared to corresponding not genetically modified wild-type plant cells.

9. A process for preparing hyaluronan comprising extracting hyaluronan from the genetically modified plant cell of claim 1.

10. (canceled)

11. A composition comprising the genetically modified plant cell of claim 1.

12. (canceled)

13. (canceled)

14. A method for preparing a composition comprising hyaluronan comprising expressing hyaluronan in the genetically modified cell of claim 1.

15. The genetically modified plant cell of claim 3, wherein said foreign nucleic acid molecule coding for a protein having the activity of a UDP-glucose dehydrogenase (UDP-Glc-DH) comprises:
a) a nucleic acid molecule coding for a protein comprising the amino acid sequence of SEQ ID NO: 5;
b) a nucleic acid molecule coding for a protein comprising a sequence at least 90%, 95%, or 98% identical to the amino acid sequence of SEQ ID NO: 5;
c) a nucleic acid molecule comprising the nucleotide sequence of SEQ ID NO 4, or the sequence complementary thereto;
d) a nucleic acid molecule at least 90%, 95%, or 98% identical to the nucleic acid sequences described under a) or c);
e) a nucleic acid molecule which hybridizes under stringent conditions at least one strand of the nucleic acid sequences described under a) or c), wherein said stringent conditions comprises 25 M sodium phosphate buffer pH 7.2, 1 mM EDTA, 7% SDS, and 65-68° C.; or
f) a nucleic acid molecule wherein the nucleotide sequence thereof differs from the sequence of the nucleic acid molecules described under a) or c) owing to the degeneration of the genetic code.

16. The plant of claim 5, wherein said plant is a corn, rice, wheat, alfalfa, rye, oats, barley, manioc, potato, tomato, switchgrass (Panicum virgalum), sago, mung beans, pas, sorghum, carrots, aubergine, radish, oilseed rape, soybeans, peanuts, cucumbers, pumpkins, melons, leek, garlic, cabbage, spinach, sweet potato, asparagus, courgettes, lettuce, artichokes, sweetcom, parsnip, scorzonera, Jerusalem artichoke, banana, sugarbeet, sugarcane, beetroot, broccoli, cabbage, onion, yellow beet, dandelion, strawberry, apple, apricot, plum, peach, grapevines, cauliflower, celery, bell peppers, swede, or rhubarb plant.

17. A process for preparing hyaluronan comprising extracting hyaluronan from the plant of claim 5.

18. A process for preparing hyaluronan comprising extracting hyaluronan from the propagation material of claim 6.

19. A process for preparing hyaluronan comprising extracting hyaluronan from the harvestable plant parts of claim 7.
